(19) United States
(12) Patent Application Publication
(10) Pub. No.: US 2024/0368677 A1
(43) Pub. Date: Nov. 7, 2024

(54) METHODS AND COMPOSITIONS FOR IN SITU DETECTION USING IMMOBILIZABLE PROBES

(71) Applicant: 10x Genomics, Inc., Pleasanton, CA (US)

(72) Inventor: Marco MIGNARDI, Sundbyberg (SE)

(21) Appl. No.: 18/653,257

(22) Filed: May 2, 2024

Related U.S. Application Data
(60) Provisional application No. 63/463,527, filed on May 2, 2023.

(51) Int. Cl. C12Q 1/682; C12Q 1/6841; C12Q 1/6844; C12Q 1/6876; G01N 1/30

(52) U.S. Cl. CPC C12Q 1/682; C12Q 1/6841; C12Q 1/6844; C12Q 1/6876; G01N 1/30

(57) ABSTRACT

The present disclosure relates in some aspects to methods and compositions for in situ detection of nucleic acids in a biological sample or matrix using nucleic acid probes that can be immobilized in the sample or matrix.

### CROSS-REFERENCE TO RELATED APPLICATION

This application claims priority to U.S. Provisional Patent Application No. 63/463,527 filed on May 2, 2023, entitled “METHODS AND COMPOSITIONS FOR IN SITU DETECTION USING IMMOBILIZABLE PROBES,” which is herein incorporated by reference in its entirety for all purposes.

### FIELD

The present disclosure relates in some aspects to methods and compositions for analysis of a target nucleic acid in a sample (e.g., in situ), such as analysis using oligonucleotides comprising attachment moieties for immobilization.

### BACKGROUND

Oligonucleotide probe-based assay methods for analysis of target nucleic acids depend on conditions affecting the stability of the hybridization complex and/or the positional stability of the hybridization complex. For example, if wash conditions are too stringent, then probe/target hybrids or amplification products thereof will be denatured, resulting in a decrease in the amount of signal in the assay. Furthermore, some methods such as isometric expansion of a sample require stabilization of target analytes to a matrix in order to preserve positional information of the target analytes in the sample (e.g., a cell or tissue sample). Thus, there is a need for increasing the spatial fidelity of target analytes (e.g., present in amplification products, such as rolling circle amplification products) during analysis of target nucleic acids in a sample (e.g., in situ analysis). Provided herein are methods and compositions that address such and other needs.

### SUMMARY

In some aspects, provided herein is a method of analyzing a biological sample, comprising: (a) contacting the biological sample with a primary immobilizable probe comprising: (1) a first nucleic acid molecule comprising (i) a first part of a split hybridization region and (ii) an overhang region comprising a primary barcode sequence; and (2) a second nucleic acid molecule comprising (i) a second part of the split hybridization region and (ii) an attachment moiety, wherein: the first part of the split hybridization region comprises a first nucleotide sequence complementary to a first portion of a region of interest in a target nucleic acid, and the second part of the split hybridization region comprises a second nucleotide sequence complementary to a second portion of the region of interest in the target nucleic acid; (b) ligating the first part to the second part using the region of interest as a template; (c) using the attachment moiety, attaching the primary immobilizable probe to the biological sample or to a matrix embedding the biological sample; and (d) detecting the primary immobilizable probe at a position in the biological sample and/or matrix.

In some aspects, provided herein is a method of analyzing a biological sample, comprising: (a) contacting the biological sample with a primary immobilizable probe comprising: (1) a first nucleic acid molecule comprising (i) a first part of a split hybridization region and (ii) an overhang region comprising a primary barcode sequence; (2) a second nucleic acid molecule comprising a second part of the split hybridization region, and (3) a splint oligonucleotide comprising (i) a splint sequence for hybridizing to the first nucleic acid molecule and the second nucleic acid molecule and (ii) an attachment moiety, wherein: the first part of the split hybridization region comprises a first nucleotide sequence complementary to a first portion of a region of interest in a target nucleic acid, and the second part of the split hybridization region comprises a second nucleotide sequence complementary to a second portion of the region of interest in the target nucleic acid; (b) ligating the first part to the second part using the region of interest as a template, and ligating the first nucleic acid molecule and second nucleic acid molecule using the splint oligonucleotide; (c) using the attachment moiety, attaching the primary immobilizable probe to the biological sample or to a matrix embedding the biological sample; and (d) detecting the primary immobilizable probe at a position in the biological sample and/or matrix.

In some embodiments, the 5′ end of the first part of the split hybridization region is ligated to the 3′ end of the second part of the split hybridization region, with or without gap filling prior to ligation; or the 5′ end of the second part of the split hybridization region is ligated to the 3′ end of the first part of the split hybridization region, with or without gap filling prior to ligation.

In some embodiments, the detecting comprises using the primary barcode sequence to generate a signal corresponding to the primary immobilizable probe. In some embodiments, the detecting comprises: rolling circle amplification (RCA) of a circular or circularized probe that is hybridized and/or directly or indirectly bound to the primary immobilizable probe; hybridization chain reaction (HCR) directly or indirectly on the primary immobilizable probe; linear oligonucleotide hybridization chain reaction (LO-HCR) directly or indirectly on the primary immobilizable probe; primer exchange reaction (PER) directly or indirectly on the primary immobilizable probe; assembly of branched structures directly or indirectly on the primary immobilizable probe; hybridization of a plurality of fluorescently labeled probes directly or indirectly on the primary immobilizable probe; or any combination thereof. In some embodiments, the detecting comprises branched DNA amplification. In some embodiments, the detecting comprises rolling circle amplification (RCA). In some embodiments, the detecting comprises: (i) hybridizing a secondary circular probe to the primary barcode sequence in the primary immobilizable probe or hybridizing a secondary circularizable probe to the primary barcode sequence in the primary immobilizable probe and circularizing the secondary circularizable probe to generate a secondary circularized probe, (ii) performing rolling circle amplification (RCA) using the secondary circular or circularized probe as a template to generate a rolling circle amplification product (RCP), and (iii) detecting the RCP at a position in the biological sample or matrix. In some embodiments, the secondary circular or circularizable probe is hybridized to the primary immobilizable probe after the primary immobilizable probe has been attached to the biological sample or to the matrix. In some embodiments, the secondary circular or circularizable probe is hybridized to the primary immobilizable probe after the first part of the split hybridization region has been ligated to the second part of the split hybridization region.

In some embodiments, the biological sample is a tissue sample and the primary immobilizable probe is attached using the attachment moiety to a matrix embedding the tissue sample. In some embodiments, the method comprises digesting the tissue sample before detecting the primary immobilizable probe at a position in the matrix. In some embodiments, the method comprises digesting the tissue sample before detecting the RCP at a position in the matrix. In some embodiments, the method can comprise digesting the tissue sample before performing RCA of the secondary circular or circularized probe. In some embodiments, the method comprises digesting the tissue sample before hybridizing the secondary circular or circularizable probe to the primary immobilizable probe.

In some embodiments, the primary immobilizable probe is a circularizable probe, and ligating the first part to the second part can circularize the primary immobilizable probe. In some embodiments, the detecting comprises performing RCA using the circularized primary immobilizable probe as a template to generate an RCP. In some embodiments, the method further comprises crosslinking the primary immobilizable probe to the target nucleic acid. In some embodiments, the secondary circular, or secondary circularizable probe and/or secondary circularized probe generated therefrom, are shorter in length than the primary immobilizable probe. In some embodiments, the secondary circular, or secondary circularizable probe and/or secondary circularized probe generated therefrom, is at least 5, at least 10, at least 20, at least 30, or at least 40 nucleotides shorter in length than the primary immobilizable probe. In some embodiments, the method comprises using the overhang region of the first nucleic acid molecule as a primer for the RCA. In some embodiments, the RCP comprises multiple copies of an RCP barcode sequence, wherein the method comprises hybridizing a detection probe to the RCP barcode sequence and detecting the detection probe or a product thereof, thereby detecting the RCP at the position in the biological sample or matrix. In some embodiments, the detection probe comprises (i) a recognition sequence that hybridizes to a sequence of the RCP and (ii) a reporter sequence for binding directly or indirectly to a reporter oligonucleotide that is associated with a detectable moiety. In some embodiments, detecting the detection probe comprises detecting the detectable moiety of the reporter oligonucleotide bound to the detection probe. In some embodiments, the detection probe is a first detection probe, and the method comprises removing the first detection probe after detecting the first detection probe, hybridizing a second detection probe to the RCP, and detecting the second detection probe. In some embodiments, the detection probe is directly associated with a detectable moiety. In some embodiments, the detectable moiety is a fluorescent label.

In some embodiments, the target nucleic acid is DNA. In some embodiments, the target nucleic acid is cDNA. In some embodiments, the target nucleic acid is RNA. In some embodiments, the target nucleic acid is mRNA. In some embodiments, the target nucleic acid is an RNA fragment. In some embodiments, the RNA fragment does not need to comprise a polyA tail.

In some embodiments, the attachment moiety is attached to an anchoring moiety in the biological sample or matrix, and the attachment moiety and the anchoring moiety is a ligand-ligand binding pair, or functional moieties that can react with each other. In some embodiments, the attachment moiety is an acrydite moiety. In some embodiments, the acrydite is a C6 methacrylate. In some embodiments, the attachment moiety is a methacrylate C6 phosphoramidite. In some embodiments, the method comprises performing one or more wash steps to remove unbound and/or nonspecifically bound probe molecules from the sample. In some embodiments, the method comprises contacting the sample with a matrix-forming material and using the matrix-forming material to form the matrix. In some embodiments, the matrix is a hydrogel matrix. In some embodiments, the matrix is functionalized with the anchoring moiety to bind covalently or non-covalently to the attachment moiety. In some embodiments, the anchoring moiety is a reactive group selected from the group consisting of acrydite, NHS ester, azide, maleimide, amine, and carboxyl groups.

In some embodiments, the biological sample is non-homogenized and optionally selected from the group consisting of a formalin-fixed, paraffin-embedded (FFPE) sample, a frozen tissue sample, and a fresh tissue sample. In some embodiments, the biological sample is permeabilized. In some embodiments, the biological sample is embedded in a matrix. In some embodiments, the matrix comprises a hydrogel. In some embodiments, the biological sample is cleared. In some embodiments, the method can comprise clearing the biological sample after attaching the primary immobilizable probe to the biological sample or matrix. In some embodiments, the RCA is performed after clearing the biological sample. In some embodiments, the clearing can comprise contacting the biological sample with a proteinase. In some embodiments, the biological sample is a tissue slice between about 1 μm and about 50 μm in thickness. In some embodiments, the tissue slice is between about 5 μm and about 35 μm in thickness. In some embodiments, the attachment moiety of the primary immobilizable probe is crosslinked to the biological sample or to a matrix embedding the biological sample.

In some aspects, provided herein is a method for sample analysis, comprising: (a) contacting a biological sample embedded in a matrix with a primary immobilizable probe comprising: (1) a first nucleic acid molecule comprising (i) a first part of a split hybridization region and (ii) an overhang region comprising a primary barcode sequence; and (2) a second nucleic acid molecule comprising (i) a second part of the split hybridization region and (ii) an attachment moiety, wherein: the first part of the split hybridization region comprises a first nucleotide sequence complementary to a first portion of a region of interest in a target nucleic acid, and the second part of the split hybridization region comprises a second nucleotide sequence complementary to a second portion of the region of interest in the target nucleic acid; (b) ligating the first part to the second part using the region of interest as a template; (c) using the attachment moiety, crosslinking the primary immobilizable probe to the biological sample or to a matrix embedding the biological sample; (d) clearing the biological sample; (d) hybridizing a secondary circular probe to the primary barcode sequence in the primary immobilizable probe or hybridizing a secondary circularizable probe to the primary barcode sequence in the primary immobilizable probe and circularizing the secondary circularizable probe to generate a secondary circularized probe; (e) performing rolling circle amplification (RCA) using the secondary circular or circularized probe as a template to generate a rolling circle amplification product (RCP); and (f) detecting the RCP at a position in the biological sample and/or matrix.

In some aspects, provided herein is a kit comprising a primary immobilizable probe, wherein: the primary immobilizable probe comprises: (1) a first nucleic acid molecule comprising (i) a first part of a split hybridization region and (ii) an overhang region comprising a primary barcode sequence; and (2) a second nucleic acid molecule comprising (i) a second part of the split hybridization region and (ii) an attachment moiety, wherein: the first part of the split hybridization region comprises a first nucleotide sequence complementary to a first portion of a region of interest in a target nucleic acid, and the second part of the split hybridization region comprises a second nucleotide sequence complementary to a second portion of the region of interest in the target nucleic acid; the first part and second part are capable of being ligated using the region of interest; and the attachment moiety is capable of being attached to a biological sample or matrix embedding the biological sample. In some embodiments, the attachment moiety is capable of being crosslinked to the biological sample or matrix embedding the biological sample. In some embodiments, the kit further comprises a secondary circular probe or circularizable probe that is capable of hybridizing to the primary barcode sequence in the primary immobilizable probe. In some embodiments, the kit further comprises the target nucleic acid.

### BRIEF DESCRIPTION OF THE DRAWINGS

The drawings illustrate certain features and advantages of this disclosure. These embodiments are not intended to limit the scope of the appended claims in any manner.

FIGS. 1A-1C show schematics illustrating example configurations of the primary immobilizable probe hybridized to the target nucleic acid. The splint oligonucleotide and/or the oligonucleotide that hybridizes to the target nucleic acid can each independently comprise or be attached to an attachment moiety, and the splint oligonucleotide can be used as a primer to initiate RCA.

FIG. 2 shows a schematic illustrating an example workflow for detection of the target nucleic acid using the primary immobilizable probe.

FIGS. 3A-3D show schematics illustrating example methods of detection of the hybridized and ligated primary immobilizable probe.

FIGS. 4A-4B show schematics illustrating an example of an attachment moiety (FIG. 4A) and crosslinkable moiety (FIG. 4B) comprised by a primary immobilizable probe or component thereof.

FIG. 5 illustrates a workflow of analysis of a biological sample (e.g., a cell or tissue sample) using an opto-fluidic instrument, according to various embodiments.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In situ detection of target analytes such as nucleic acids in biological samples using microscopic imaging can provide valuable information regarding analyte abundance and localization in situ. Thus, in situ assays are important tools, for example, for understanding the molecular basis of cell identity and developing treatment for diseases.

In situ analysis in certain biological samples can be affected based on a number of factors, including the source of the sample and the method by which the sample is prepared for analysis. For example, fixed biological samples, such as Formalin-Fixed Paraffin-Embedded (FFPE) samples, are prone to degradation of target analytes such as transcripts (e.g. mRNA) and high background, affecting the detection and analysis of target analytes using certain methods. For example, methods that rely on tiling of individual target nucleic acid transcripts with multiple probes (e.g. using single-molecule fluorescence in situ hybridization (smFISH)) can be hindered in FFPE samples due to transcript fragmentation, such that many of the probes cannot bind the target transcript, thereby negatively impacting the detection signal. In some methods of smFISH, transcripts may be captured and embedded in a gel (e.g. a polyacrylamide gel) prior to detection, which can reduce background and increase signal-to-noise ratio. However, in many cases, transcripts are captured using poly(A) capture, which requires transcript integrity that is not present in FFPE samples.

Provided herein are methods and compositions that relate to detection of nucleic acid molecules, including nucleic acid molecules that may be short, or shortened and/or fragmented, for example as a result of tissue preparation (e.g. in FFPE samples). Thus, in some aspects, the methods herein provide the ability to perform in situ detection and analysis of target analytes in fixed biological samples, such as FFPE samples. In some embodiments, the methods comprise detecting a signal that is indicative of the presence and/or position of the target nucleic acid in the sample.

In some aspects, provided herein are methods involving the use of immobilizable probes functionalized with an attachment moiety for attachment to a biological sample or matrix, for analysis of target nucleic acid(s) in a sample (e.g., a cell or a biological sample, such as a tissue sample). In some embodiments, the immobilizable probe can also be crosslinked to the target nucleic acid. Also provided are polynucleotides, sets of polynucleotides, compositions, kits, systems, and devices for use in accordance with the provided methods. In some aspects, the provided methods and compositions can be applied to maintain the spatial fidelity of target nucleic acids and/or immobilizable probes corresponding thereto during downstream analyses (e.g., in situ analysis).

In some aspects, provided herein is a method of analyzing a biological sample. In some embodiments, the method comprises contacting the biological sample with a primary immobilizable probe. In some embodiments, the primary immobilizable probe comprises a primary barcode sequence, an attachment moiety, and a split hybridization region. In some embodiments, the split hybridization region comprises a first part and a second part. In some embodiments, the first part of the split hybridization region comprises a first nucleotide sequence complementary to a first portion of a region of interest in a target nucleic acid, and the second part of the split hybridization region comprises a second nucleotide sequence complementary to a second portion of the region of interest in the target nucleic acid. In some embodiments, the method comprises ligating the first part to the second part using the region of interest as a template. In some embodiments, the method comprises ligating the first part of the split hybridization region to the second part of the split hybridization region to form a ligated primary immobilizable probe. In some embodiments, the method comprises using the attachment moiety to attach (e.g. crosslink) the ligated primary immobilizable probe to the biological sample or to a matrix embedding the biological sample. In some embodiments, the method comprises detecting the ligated primary immobilizable probe at a position in the biological sample and/or matrix. In some embodiments, the primary immobilizable probe comprises a first nucleic acid molecule and a second nucleic acid molecule. Thus, in some embodiments, the primary immobilizable probe comprises a plurality of nucleic acid molecules (e.g., the primary immobilizable probe is a probe set). In some embodiments, the first nucleic acid molecule comprises (i) the first part of the split hybridization region and (ii) an overhang region comprising the primary barcode sequence; and the second nucleic acid molecule comprises (i) the second part of the split hybridization region and (ii) the attachment moiety. In some embodiments, the two nucleic acid molecules hybridize to a first and second portion of the region of interest in the target nucleic acid. In some embodiments, the target nucleic acid is a specific RNA molecule.

In some embodiments, the primary immobilizable probe comprises a first nucleic acid molecule, a second nucleic acid molecule, and a splint oligonucleotide. For example, the first nucleic acid molecule comprises the first part of the split hybridization region and an overhang region comprising the primary barcode sequence; and the second nucleic acid molecule comprises the second part of the split hybridization region and the splint oligonucleotide comprises the attachment moiety. In some embodiments, the splint oligonucleotide is used to ligate the first nucleic acid molecule to the second nucleic acid molecule. In some embodiments, the two nucleic acid molecules hybridize to a first and second portion of the region of interest in the target nucleic acid. In some embodiments, the target nucleic acid is a specific RNA molecule. In some embodiments, the splint oligonucleotide is used as a rolling circle amplification (RCA) primer for an RCA reaction that uses the ligated first and second nucleic acid molecules as a circular template for the RCA.

In some embodiments, the detecting comprises using the primary barcode sequence to generate a signal corresponding to the ligated primary immobilizable probe. In some embodiments, the detecting comprises any suitable method for detection, such as rolling circle amplification (RCA) or assembly of branched structures (e.g. branched DNA amplification). In some embodiments, the detecting comprises: (i) hybridizing a secondary circular probe to the primary barcode sequence in the ligated primary immobilizable probe or hybridizing a secondary circularizable probe to the primary barcode sequence in the ligated primary immobilizable probe and circularizing the secondary circularizable probe to generate a secondary circularized probe, (ii) performing rolling circle amplification (RCA) using the secondary circular or circularized probe as a template to generate a rolling circle amplification product (RCP), and (iii) detecting the RCP at a position in the biological sample or matrix.

In some aspects, the methods include hybridizing an immobilizable probe (e.g. the primary immobilizable probe) to the target nucleic acid. In some embodiments, the immobilizable probe comprises an attachment moiety that is attached (e.g. crosslinked) to the biological sample or a matrix embedding the biological sample. In some embodiments, the sample is embedded in a gel (e.g. acrylamide gel) and attached (e.g. crosslinked). In some embodiments, probes containing an attachment moiety (e.g. probes containing acrydite groups) will be captured (e.g. will remain in the sample) after the gel is polymerized and the tissue is digested. In some embodiments, the captured probes can then be detected and analyzed, for example by sequential hybridization, detection, and decoding.

Detection can occur by any suitable method, including methods comprising signal amplification. For example, in some embodiments, the barcode of the ligated primary immobilizable probe is hybridized by a secondary circular or circularized probe that is used as an RCA template to generate an RCP for detection. In other embodiments, the barcode of the ligated primary immobilizable probe is detected using a branched hybridization complex.

In some embodiments, the attachment moiety is any suitable attachment moiety, such as an acrydite group or another group that is attached (e.g. crosslinked) to a gel for embedding the sample. In some embodiments, the attachment moiety is an acrydite moiety. In some embodiments, the acrydite is a C6 methacrylate. In some embodiments, the attachment moiety is a methacrylate C6 phosphoramidite.

In some embodiments, the primary immobilizable probe comprises a barcode sequence (e.g. a gene-specific sequence), that corresponds to the target nucleic acid. In some embodiments, the barcode sequence is used to detect, and determine the identity of, the target nucleic acid. For example, in some embodiments, the barcode sequence can be identified by sequential hybridization, detection and decoding methods, such as any described herein.

In some embodiments, the attaching (e.g. crosslinking) stabilizes the position of the ligated primary immobilizable probe in the sample and/or matrix, and/or allows the primary immobilizable probe to remain present in the sample during subsequent processing steps. In some embodiments, once the immobilizable probe is hybridized and attached at a position in the biological sample, a signal generated from the detectable probe is indicative of the presence and position of the target nucleic acid in the biological sample before the method was performed, and the target nucleic acid need not remain present in the biological sample in order to detect its original presence and position. Thus, in some embodiments, following hybridization and attaching (e.g. crosslinking) of the immobilizable probe, the sample undergoes further processing steps that may or may not preserve the integrity and/or position of the target nucleic acid in the sample. For example, in some embodiments, the sample may be digested and/or cleared, e.g. with a proteinase. In some embodiments, the immobilizable probe is detected after the one or more further processing steps. In some embodiments, the primary immobilizable probe is crosslinked to the target nucleic acid.

In some aspects, the compositions and methods provided herein are advantageous for producing signals with high specificity. For example, in some aspects, a signal is only generated from a nucleic acid when both the first and second portions of the region of interest are present in the nucleic acid, thereby allowing the first part and second part of the split hybridization region to hybridize and ligate using the nucleic acid as template. For example, in some embodiments, the primary immobilizable probe comprises a first nucleic acid molecule and a second nucleic acid molecule, wherein: the first nucleic acid molecule comprises (i) the first part of the split hybridization region and (ii) an overhang region comprising the primary barcode sequence; and the second nucleic acid molecule comprises (i) the second part of the split hybridization region and (ii) the attachment moiety. In such embodiments, the ligated primary immobilizable probe will comprise both a barcode sequence for detection, and an attachment moiety for attaching (e.g. crosslinking) to the sample. Conversely, an unhybridized and/or unligated first nucleic acid molecule comprising the barcode sequence for detection will not be attached to the sample or matrix via a second nucleic acid molecule, and will be removed from the sample. On the other hand, an unligated second nucleic acid molecule will not be detectable, since it does not comprise a barcode sequence for detection. In other embodiments, the primary immobilizable probe can be a circularizable probe. In such embodiments, hybridization and ligation of the first and second part of the split hybridization region (e.g. circularization of the primary immobilizable probe) can allow the primary immobilizable probe to remain present in the sample for detection during subsequent steps. For example, in some embodiments, the circularized primary immobilizable probe can remain hybridized to the target nucleic acid during washes under conditions that are sufficiently stringent to remove uncircularized primary immobilizable probes. In some embodiments, circularization can protect the primary immobilizable probe from degradation, for example by digestion.

### II. Polynucleotides and Hybridization Complexes

Disclosed herein in some aspects are nucleic acid probes and/or probe sets, such as primary immobilizable probes, that are introduced into a cell or used to otherwise contact a biological sample such as a tissue sample. The probes may comprise any of a variety of entities that can hybridize to a nucleic acid, typically by Watson-Crick base pairing, such as DNA, RNA, LNA, PNA, etc., depending on the application. The nucleic acid probe(s) typically contain a hybridization region that is able to bind to at least a portion of a target nucleic acid, in some embodiments specifically. The nucleic acid probe may be able to bind to a specific target nucleic acid (e.g., an mRNA, or other nucleic acids as discussed herein). In some embodiments, the nucleic acid probes may be detected using a detectable label, and/or by using detectably labeled nucleic acid probes able to bind to the nucleic acid probes or amplification products thereof, directly or via an intermediate probe. In some embodiments, the nucleic acid probes are compatible with one or more biological and/or chemical reactions. For instance, a primary nucleic acid probe disclosed herein can serve as a template or primer for a polymerase (e.g., for rolling circle amplification), a template or substrate for a ligase, a substrate for a click chemistry reaction, and/or a substrate for a nuclease (e.g., endonuclease for cleavage).

### A. Primary Immobilizable Probes

In some aspects, provided herein are primary immobilizable probes for detection of target nucleic acids in a biological sample or matrix. In some aspects, the primary immobilizable probes comprise a split hybridization region, an attachment moiety, and a primary barcode sequence, which can be present in various configurations. In some aspects, the primary immobilizable probe can be hybridized to a target nucleic acid, immobilized in the biological sample or matrix, and detected.

In some aspects, provided herein is a method of analyzing a biological sample. In some embodiments, the method comprises contacting the biological sample with a primary immobilizable probe. In some embodiments, the primary immobilizable probe comprises a primary barcode sequence, an attachment moiety, and a split hybridization region. In some embodiments, the split hybridization region comprises a first part and a second part. In some embodiments, a primary immobilizable probe, comprises two separate nucleic acid molecules, e.g., a first nucleic acid molecule and a second nucleic acid molecule. In some instances, the first nucleic acid molecule comprises the first part of the split hybridization region and the second nucleic acid molecule comprises the second part of the split hybridization region. In some embodiments, the first part of the split hybridization region comprises a first nucleotide sequence complementary to a first portion of a region of interest in a target nucleic acid, and the second part of the split hybridization region comprises a second nucleotide sequence complementary to a second portion of the region of interest in the target nucleic acid. In some embodiments, the method comprises ligating the first part to the second part using the region of interest as a template to form a ligated primary immobilizable probe. In some embodiments, the method comprises using the attachment moiety to attach (e.g. crosslink) the ligated primary immobilizable probe to the biological sample or to a matrix embedding the biological sample. In some embodiments, the method comprises detecting the ligated primary immobilizable probe at a position in the biological sample and/or matrix. In some embodiments, the primary immobilizable probe comprises a first nucleic acid molecule and a second nucleic acid molecule. In some embodiments, the first nucleic acid molecule comprises (i) the first part of the split hybridization region and (ii) an overhang region comprising the primary barcode sequence. In some embodiments, the second nucleic acid molecule comprises (i) the second part of the split hybridization region and (ii) the attachment moiety.

Examples of primary immobilizable probe configurations and methods for their use in detection of target nucleic acids are described in connection with the figures in detail below.

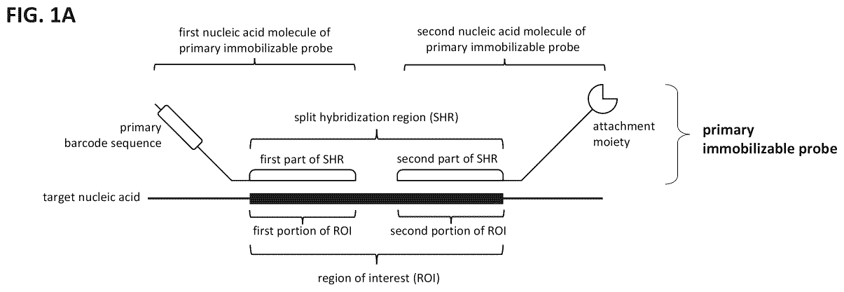

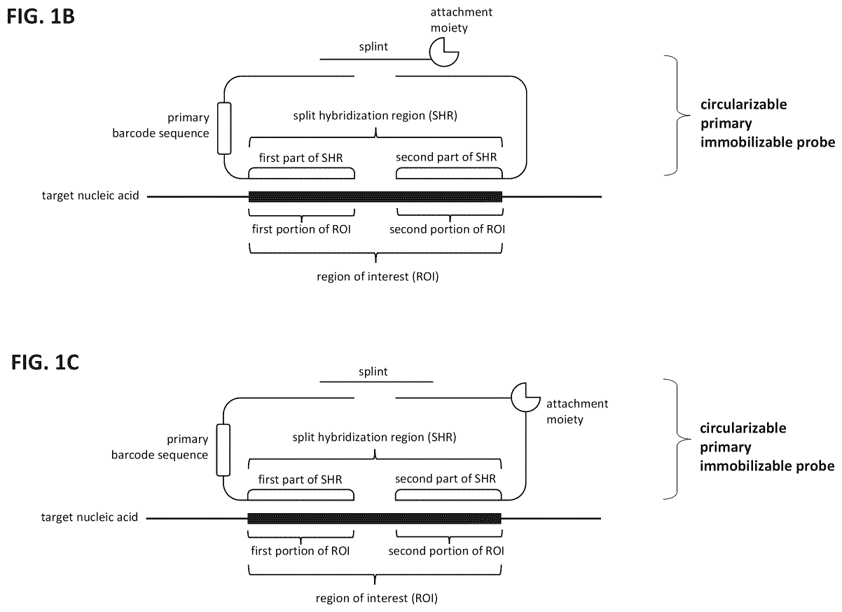

FIGS. 1A-1C show schematics illustrating example configurations of the primary immobilizable probe hybridized to the target nucleic acid. The primary immobilizable probe comprises a split hybridization region comprising a first part that hybridizes to a first portion of a region of interest in a target nucleic acid, and a second part that hybridizes to a second portion of the region of interest in the target nucleic acid. In some embodiments, the first part and the second part are from two separate nucleic acid molecules. The first part and second part are configured to be ligated using the region of interest as template, with or without gap filling prior to ligation. The primary immobilizable probe further comprises an attachment moiety. The attachment moiety can be attached (e.g. crosslinked) to the biological sample or to a matrix embedding the biological sample. The attachment moiety can be any suitable attachment moiety, such as any described herein, for example in Section III. For example, the attachment moiety can be an acrydite moiety. In some embodiments, the matrix is a polyacrylamide gel matrix. In some embodiments, attaching (e.g. crosslinking) of the primary immobilizable probe allows for further processing steps to occur, such as digestion and washing of the sample, while preserving the position of the primary immobilizable probe, for example for detection in subsequent steps. The primary immobilizable probe further comprises a primary barcode sequence. The primary barcode sequence can facilitate detection by any suitable method, such as rolling circle amplification, and/or any of the methods described or illustrated in the figures herein.

FIG. 1A shows a primary immobilizable probe comprising a first nucleic acid molecule and a second nucleic acid molecule. The first nucleic acid molecule comprises (i) the first part of the split hybridization region and (ii) an overhang region comprising the primary barcode sequence. The second nucleic acid molecule comprises (i) the second part of the split hybridization region and (ii) the attachment moiety. In some embodiments, ligation of split hybridization region leads to the formation of a single molecule (i.e., a ligated primary immobilizable probe) comprising both the attachment moiety for attaching (e.g. crosslinking) to the biological sample or matrix, and the primary barcode sequence for detection. In the illustrated configuration, it can be seen that in some embodiments in connection with methods provided herein, the primary immobilizable probe will only be detected when both the first and second nucleic acid of the primary immobilizable hybridize to the region of interest and are ligated (i.e., when the ligated primary immobilizable probe is formed). For example, the first nucleic acid molecule alone (e.g. an unligated first nucleic acid molecule) lacks the attachment moiety, and will be removed from the sample during processing and/or washing steps. The second nucleic acid molecule alone (e.g. an unligated second nucleic acid molecule) lacks the primary barcode sequence, and thus will not be detected by the methods that rely on detection of the primary barcode sequence. Unhybridized and/or unligated primary immobilizable probes or components thereof may also be washed out of the sample at any suitable step, including before attaching (e.g. crosslinking) and detection.

FIGS. 1B-1C show example configurations of a primary immobilizable probe that is circularizable. In some embodiments, the primary immobilizable probe comprises a first nucleic acid molecule, a second nucleic acid molecule, and a splint oligonucleotide, wherein the splint oligonucleotide comprises a splint sequence for hybridizing to the first nucleic acid molecule and the second nucleic acid molecule; and the method comprises ligating the first nucleic acid molecule and second nucleic acid molecule using the splint oligonucleotide.

FIG. 1B illustrates an embodiment of a circularizable primary immobilizable probe comprising three nucleic acid molecules, including a first nucleic acid molecule and a second nucleic acid molecule comprising the first and second part of the split hybridization region, respectively, and a splint oligonucleotide comprising the attachment moiety. In some cases, the first nucleic acid molecule comprises the primary barcode sequence, and the splint oligonucleotide comprises the attachment moiety. As shown in FIG. 1B, in some embodiments, ligation of the first and second part of the split hybridization region using the target nucleic acid as template and ligation of the first nucleic acid molecule and second nucleic acid molecule at a region not hybridized to the target nucleic acid using the splint oligonucleotide as template leads to circularization of the circularizable primary immobilizable probe.

FIG. 1C illustrates an embodiment of a circularizable primary immobilizable probe comprising two or more nucleic acid molecules. In FIG. 1C, the circularizable primary immobilizable probe comprises a first nucleic acid molecule comprising (i) the first part of the split hybridization region and (ii) an overhang region comprising the primary barcode sequence; and a second nucleic acid molecule comprising (i) the second part of the split hybridization region and (ii) the attachment moiety. The circularizable primary immobilizable probe may further comprise a splint oligonucleotide that serves as a template for ligation of the ends of the first and second nucleic acid molecules of the circularizable primary immobilizable probe not comprising the split hybridization region. In some embodiments, the ends of the first and second nucleic acid molecules of the circularizable primary immobilizable probe not comprising the split hybridization region constitute a first and second segment of a splint oligonucleotide hybridization region.

In the illustrated example configurations of circularizable primary immobilizable probes shown in FIG. 1B and FIG. 1C, the splint oligonucleotide hybridizes to ends of the first and second nucleic acid molecules of the primary immobilizable probe (e.g. splint oligonucleotide hybridization region). In some embodiments, the splint oligonucleotide hybridizes the first nucleic acid molecule and the second nucleic acid molecule of the primary immobilizable probe via a splint sequence. In some embodiments, the splint oligonucleotide hybridizes to a 5′ end of the first nucleic acid molecule and a 3′ end of the second nucleic acid molecule. In other embodiments, the splint oligonucleotide hybridizes to a 3′ end of the first nucleic acid molecule and a 5′ end of the second nucleic acid molecule. In some embodiments, the splint oligonucleotide hybridizes to ends of the first and second nucleic acid molecules that do not comprise the portions of the split hybridization regions. Thus, in some embodiments, the target nucleic acid serves as a template for ligating the two ends of the first and second nucleic acid molecules comprising the split hybridization region, and the splint oligonucleotide serves as a template for ligating the other two ends of the first and second nucleic acid molecule that are not hybridized to the target nucleic acid, thereby forming a circularized molecule from the first and second nucleic acid molecules. The splint may serve as a template for ligation with or without gap filling prior to ligation.

In some embodiments, the splint oligonucleotide hybridizes specifically to a first and second nucleic acid molecule of a primary immobilizable probe. In some embodiments, a plurality of primary immobilizable probes for detecting different target nucleic acids and/or regions of interest is used. In such embodiments, one or more different primary immobilizable probes may comprise common sequences for hybridizing to a common splint oligonucleotide that serves as a template for ligating the different primary immobilizable probes. Thus, a single splint oligonucleotide can facilitate circularization of various primary immobilizable probes. In other embodiments, the different primary immobilizable probes comprises different sequences for hybridizing to different splint oligonucleotides.

In some embodiments, the splint oligonucleotide hybridization region of the circularizable primary immobilizable probe is between about 5 and about 60 nucleotides in length, e.g., between about 5 and about 50, between about 5 and about 40, between about 5 and about 30, between about 5 and about 25, between about 5 and about 15, between about 10 and about 50, between about 10 and about 40, between about 10 and about 30, or between about 20 and about 50 nucleotides in length. In some embodiments, the first segment and/or second segment of the splint oligonucleotide hybridization region is between about 5 and about 60 nucleotides in length, e.g., between about 5 and about 50, between about 5 and about 40, between about 5 and about 30, between about 5 and about 25, between about 5 and about 15, between about 10 and about 50, between about 10 and about 40, between about 10 and about 30, or between about 20 and about 50 nucleotides in length.

In some embodiments, the splint oligonucleotide hybridization region is ligated. In some embodiments, the first segment of the splint oligonucleotide hybridization region is ligated to the second segment of the splint oligonucleotide hybridization region, using the splint oligonucleotide as template. In some embodiments, the 5′ end of the first segment of the splint oligonucleotide hybridization region is ligated to the 3′ end of the second segment of the splint oligonucleotide hybridization region. In some embodiments, the 5′ end of the second segment of the splint oligonucleotide hybridization region is ligated to the 3′ end of the first segment of the splint oligonucleotide hybridization region. In some embodiments, the ligation does not require gap filling prior to ligation. For example, the first and second segment of the splint oligonucleotide hybridization region can hybridize to directly adjacent sequences in the splint oligonucleotide, thus being configured to be ligated without gap filling. In other embodiments, the ligation includes gap filling prior to ligation. For example, the first and second segment of the splint oligonucleotide hybridization region can hybridize to sequences in the splint oligonucleotide that are separated by one or more intervening nucleotides. In this example, a polymerase could be used for gap-filling, e.g. to extend the 3′ end of the first or second segment of the splint oligonucleotide hybridization region using the splint oligonucleotide as template, prior to ligation.

In the illustrated example configurations of circularizable primary immobilizable probes shown in FIG. 1B and FIG. 1C, it can be seen that in some embodiments in connection with methods provided herein, the circularizable primary immobilizable probe will only be detected when the circularizable primary immobilizable probe is circularized, for example by ligation of the split hybridization region and ligation of the splint oligonucleotide hybridization region. For example, in some embodiments, an unhybridized and/or uncircularized primary immobilizable probe or component thereof may be washed out of the sample prior to attaching (e.g. crosslinking) and detection steps. In another example, uncircularized primary immobilized probes or components thereof may be removed or degraded enzymatically prior to detection, e.g. with an exonuclease. In another example, detection of the primary immobilizable probe may be achieved by rolling circle amplification using the circularized primary immobilizable probe as template. In this example, an uncircularized primary immobilizable probe or component thereof could not serve as a template for RCA, and would not be detected. When the circularizable primary immobilizable probe comprises more than one nucleic acid, for example as shown in FIG. 1B and FIG. 1C, in some embodiments, the individual unligated nucleic acid molecules of the probe will not be detected. For example, the first nucleic acid molecule alone (e.g. an unligated first nucleic acid molecule) lacks the attachment moiety, and will be removed from the sample during processing and/or washing steps. The second nucleic acid molecule alone (e.g. an unligated second nucleic acid molecule) lacks the primary barcode sequence, and thus will not be detected by the methods that rely on detection of the primary barcode sequence. Unhybridized and/or unligated primary immobilizable probes or components thereof may also be washed out of the sample at any suitable step, including before attaching (e.g. crosslinking) and detection.

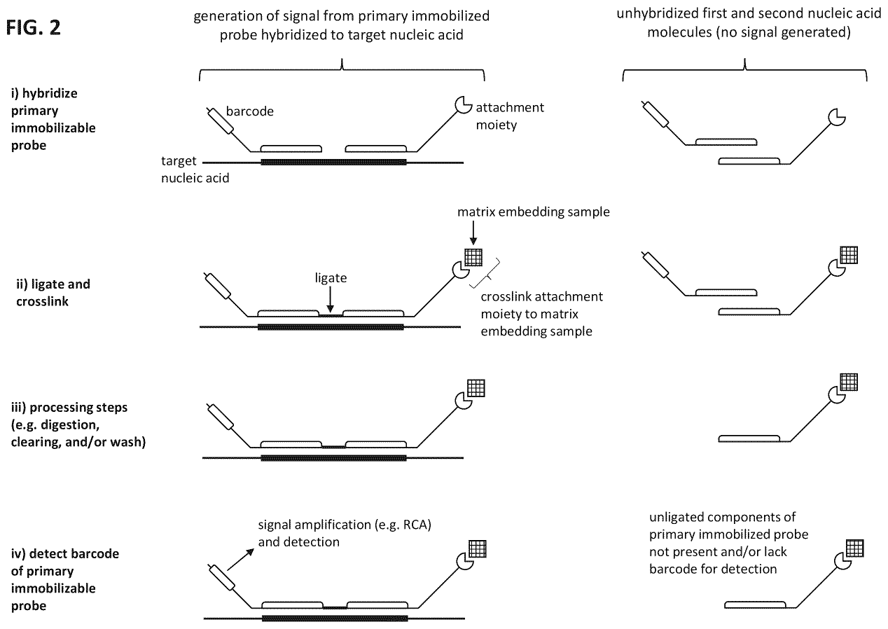

FIG. 2 shows a schematic illustrating an example workflow for detection of the target nucleic acid using a primary immobilizable probe. In panel (i), the primary immobilizable probe comprising a first and second nucleic acid molecule (e.g. as illustrated in FIG. 1A), is hybridized to the region of interest in the target nucleic acid. Unhybridized first and second nucleic acid molecules are shown on the right. In panel (ii), the split hybridization region of the primary immobilizable probe is ligated using the target nucleic acid as template, and the attachment moiety is attached (e.g. crosslinked) to a matrix embedding the biological sample. In panel (iii), the sample undergoes processing steps such as digestion, tissue clearing, and or washing. Unhybridized or unligated components of the primary immobilizable probe are removed. In panel (iv), a signal is generated from the primary barcode sequence of the primary immobilizable probe, for example by RCA. As shown on the right, no signal is generated from unhybridized and/or unligated components of the primary immobilizable probe. Additional wash steps may be performed at earlier steps (e.g. after hybridization and/or ligation), such that unligated/unhybridized nucleic acids comprising the attachment moiety are also removed prior to attaching (e.g. crosslinking), and do not remain in the sample.

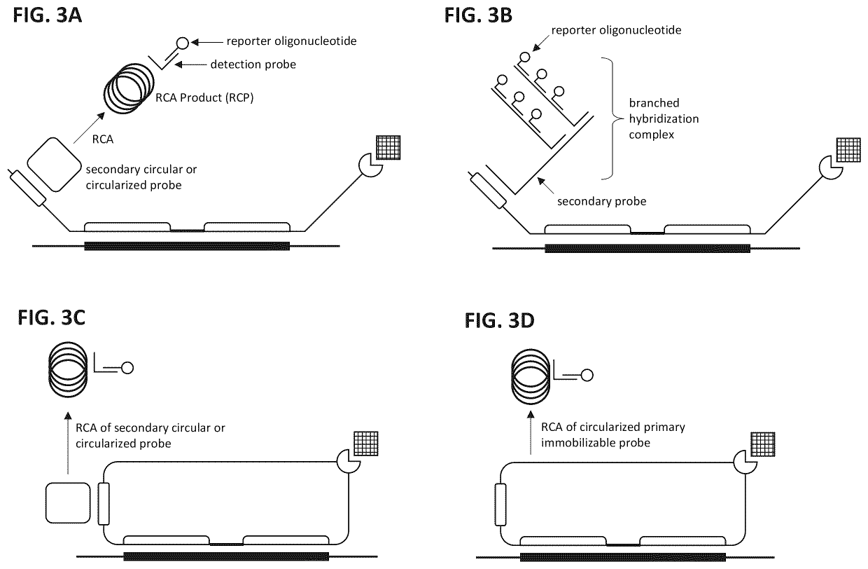

FIGS. 3A-3D show schematics illustrating example methods of detection of the hybridized and ligated primary immobilizable probe. FIG. 3A shows detection of a ligated primary immobilizable probe that is linear, using rolling circle amplification (RCA). A secondary circular probe or circularized probe hybridized to the primary barcode sequence is used as a template for RCA to produce an RCA product (RCP). The RCP is detected using a detection probe that hybridizes to a barcode sequence (e.g. an RCP barcode sequence) in the RCP. Although not shown in the figure, the RCP can comprise multiple copies of the RCP barcode sequence, such that multiple detection probes hybridize to the RCP, resulting in an amplified signal. The detection probe is hybridized to a reporter oligonucleotide comprising a detectable moiety (e.g. a fluorophore). Alternatively, the detection probe can be directly associated with the detectable moiety. The detectable moiety is used to generate and detect a signal associated with the target nucleic acid at a position in the biological sample. FIG. 3B shows detection of a ligated primary immobilizable probe that is linear, using a branched hybridization complex. A secondary probe is hybridized to the primary barcode sequence, and the secondary probe is hybridized directly or indirectly by one or more additional higher order probes to form a branched hybridization complex. The branched hybridization complex is hybridized by a plurality of reporter oligonucleotides for detection. The reporter oligonucleotides can also hybridize directly to the secondary probe. The configuration shown in FIG. 3B is representative of detection using a branched hybridization complex, and any suitable configuration of a branched hybridization complex may be used in connection with the methods herein. FIG. 3C shows detection of a ligated primary immobilizable probe that is circularized, using rolling circle amplification (RCA). A secondary circular probe or circularized probe hybridized to the primary barcode sequence is used as a template for RCA to produce an RCA product (RCP), which is detected, for example as in FIG. 3A. FIG. 3D shows detection of a ligated primary immobilizable probe that is circularized, using RCA. The circularized primary immobilizable probe is itself used as the template for RCA.

In some embodiments, the primary immobilizable probe comprises a hybridization region that hybridizes to a target nucleic acid in the biological sample. In some embodiments, the hybridization region is a split hybridization region. In some cases, the hybridization region comprises one or more sequences that are complementary to one or more sequences in the target nucleic acid. In some embodiments, the hybridization region is complementary to a sequence in the target nucleic acid. For example, in some embodiments, the first part of the split hybridization region is complementary to a first portion of a region of interest in the target nucleic acid, and the second part of the split hybridization region is complementary to a second portion of the region of interest in the target nucleic acid. In some embodiments, the hybridization region is complementary to a sequence of a target nucleic acid of interest or a subset of target nucleic acids in the biological sample.

In some embodiments, the hybridization region is between about 5 and about 60 nucleotides in length, e.g., between about 5 and about 50, between about 5 and about 40, between about 5 and about 30, between about 5 and about 25, between about 5 and about 15, between about 10 and about 50, between about 10 and about 40, between about 10 and about 30, or between about 20 and about 50 nucleotides in length. In some embodiments, the first part and/or second part of the split hybridization region is between about 5 and about 60 nucleotides in length, e.g., between about 5 and about 50, between about 5 and about 40, between about 5 and about 30, between about 5 and about 25, between about 5 and about 15, between about 10 and about 50, between about 10 and about 40, between about 10 and about 30, or between about 20 and about 50 nucleotides in length.

In some embodiments, the split hybridization region is ligated. In some embodiments, the first part of the split hybridization region is ligated to the second part of the split hybridization region, using the region of interest as template. In some embodiments, the 5′ end of the first part of the split hybridization region is ligated to the 3′ end of the second part of the split hybridization region. In some embodiments, the 5′ end of the second part of the split hybridization region is ligated to the 3′ end of the first part of the split hybridization region. In some embodiments, the ligation does not require gap filling prior to ligation. For example, the first and second part of the split hybridization region can hybridize to directly adjacent sequences in the region of interest in the target nucleic acid, thus being configured to be ligated without gap filling. In other embodiments, the ligation includes gap filling prior to ligation. For example, the first and second part of the split hybridization region can hybridize to sequences in the region of interest that are separated by one or more intervening nucleotides. In this example, a polymerase could be used for gap-filling, e.g. to extend the 3′ end of the first or second part of the split hybridization region using the region of interest as template, prior to ligation.

In some embodiments, the primary immobilizable probe comprises an attachment moiety for attaching (e.g. crosslinking) the primary immobilizable probe to the biological sample or matrix, such as a matrix embedding the biological sample. In some embodiments, the attachment moiety is any suitable attachment moiety described herein. In some embodiments, the attachment moiety is attached to an anchoring moiety in the biological sample or matrix. In some embodiments, the attachment moiety and the anchoring moiety are a ligand-ligand binding pair. In some embodiments, the attachment moiety and anchoring moiety are functional moieties that can react with each other. In some embodiments, the attachment moiety is an acrydite moiety. In some embodiments, the acrydite is a C6 methacrylate. In some embodiments, the attachment moiety is a methacrylate C6 phosphoramidite. In some embodiments, the method comprises contacting the sample with a matrix-forming material and using the matrix-forming material to form the matrix. In some embodiments, the matrix is a hydrogel matrix. In some embodiments, the matrix is functionalized with the anchoring moiety to bind covalently or non-covalently to the attachment moiety. In some embodiments, the anchoring moiety is a reactive group selected from the group consisting of acrydite, NHS ester, azide, maleimide, amine, and carboxyl groups.

In some embodiments, formation of the matrix may involve more than one polymerization steps. In some embodiments, the matrix can comprise and/or be functionalized with more than one anchoring moiety. In some embodiments, the different polymerization steps allow for the functionalization of the matrix with different anchoring moieties. In some embodiments, different anchoring moieties of the matrix are used for attachment of different components, such as various analytes, the target nucleic acid, the primary immobilizable probe, or products thereof.

In some embodiments, the primary immobilizable probe is crosslinked to the biological sample or matrix embedding the biological sample via the attachment moiety. In some embodiments, the primary immobilizable probe is also crosslinked to the target nucleic acid, thus also immobilizing the target nucleic acid. In some embodiments, the crosslinking occurs between the hybridization region of the primary immobilizable probe and the hybridized target nucleic acid. In some embodiments, the primary immobilizable probe comprises a crosslinkable moiety for crosslinking to the target nucleic acid. In some embodiments, the crosslinkable moiety is configured to crosslink to a nucleobase of the hybridized target nucleic acid. In some embodiments, the crosslinkable moiety is a modified nucleoside in the primary immobilizable probe, or is connected to a nucleotide residue in the hybridization region of the primary immobilizable probe. Exemplary crosslinkable moieties and attachment moieties are described in more detail in Sections III and IV below. In some embodiments, the crosslinkable moiety does not react with guanines of the target nucleic acid (e.g., the N7 position of guanines). However, as described herein, the methods do not need to include crosslinking the primary immobilizable probe to the target nucleic acid.

In some embodiments, a primary immobilizable probe provided herein comprises an attachment moiety, such as any suitable attachment moiety provided herein. In some embodiments, the attachment moiety is comprised by any suitable part of the primary immobilizable probe (e.g. the first nucleic acid molecule, the second nucleic acid molecule, or the splint oligonucleotide). In some embodiments, a primary immobilizable probe provided herein is functionalized with an attachment moiety. In some embodiments, the attachment moiety is attached (e.g. crosslinked) to the biological sample or matrix (e.g. a matrix embedding the biological sample), thereby immobilizing the primary immobilizable probe.

In some embodiments, a splint oligonucleotide of the primary immobilizable probe provided herein comprises an attachment moiety, such as any suitable attachment moiety provided herein. In some embodiments, the splint oligonucleotide is used as a primer to initiate amplification, and the attachment moiety is attached (e.g., crosslinked) to the biological sample or matrix (e.g., a matrix embedding the biological sample), thereby immobilizing the amplification product. In some embodiments, the amplification product is an RCA product and is anchored to the biological sample or matrix via the splint oligonucleotide.

In some embodiments, immobilizing the primary immobilizable probe in the biological sample or matrix allows the primary immobilizable probe to be detected at the position at which it is immobilized, including after subsequent processing steps, such as washing, tissue clearing, and/or enzymatic processing steps, such as digestion. In some aspects, once the primary immobilizable probe is hybridized to the target nucleic acid, ligated, and attached (e.g. crosslinked), the target nucleic acid need not remain present and/or hybridized to the immobilizable probe complex in order to detect the presence and/or original position of the target nucleic acid in the biological sample or matrix. Thus, in some embodiments, the primary immobilizable probe serves as an indicator of the presence and/or position of the target nucleic acid in the original biological sample, even after the target nucleic acid has been removed and/or de-hybridized from the primary immobilizable probe. In some embodiments, a portion of the primary immobilizable probe becomes attached and is detected. For example, in some embodiments, the splint oligonucleotide of the primary immobilizable probe is attached to the matrix via the attachment moiety, and the splint oligonucleotide is amplified (e.g. by serving as a primer for RCA) and detected, with the splint oligonucleotide remaining attached to the matrix, thus anchoring the resulting amplification product (e.g. RCA product).

In some embodiments, the primary immobilizable probe is detected at a position in the biological sample or matrix. In some embodiments, the detection is by any suitable method, such as any described herein. In some embodiments, the primary immobilizable probe is detected using the primary barcode sequence of the primary immobilizable probe. In some embodiments, the primary immobilizable probe is used to generate a product that is detected, for example as shown in FIGS. 3A-3D. In some embodiments, the detecting comprises any suitable method for detection, including rolling circle amplification (RCA) of a circular or circularized probe that is hybridized directly or indirectly to the primary immobilizable probe; hybridization chain reaction (HCR) directly or indirectly on the primary immobilizable probe; linear oligonucleotide hybridization chain reaction (LO-HCR) directly or indirectly on the primary immobilizable probe; primer exchange reaction (PER) directly or indirectly on the primary immobilizable probe; assembly of branched structures directly or indirectly on the primary immobilizable probe; hybridization of a plurality of fluorescently labeled probes directly or indirectly on the primary immobilizable probe; or any combination thereof.

In some embodiments, RCA is performed using as template a secondary circular or circularized probe that is hybridized to the primary immobilizable probe (e.g. as shown in FIG. 3A or FIG. 3C). In some embodiments, the secondary circular, or secondary circularizable probe and/or secondary circularized probe generated therefrom, is shorter in length than the primary immobilizable probe. In some embodiments, the secondary circular, or secondary circularizable probe and/or secondary circularized probe generated therefrom, is at least 5, at least 10, at least 20, at least 30, or at least 40 nucleotides shorter in length than the primary immobilizable probe.

In some embodiments, RCA is performed using as template a circularized primary immobilizable probe (e.g. as shown in FIG. 3D). In some embodiments, the primary immobilizable probe is a circularizable probe, and wherein ligating the first part to the second part circularizes the primary immobilizable probe. In some embodiments, the detecting comprises performing RCA using the circularized primary immobilizable probe as a template to generate an RCP.

In some embodiments, an RCA primer is hybridized to the template for RCA, and the primer is extended by polymerization in the RCA reaction to generate the RCP. In some embodiments, the overhang region of the first nucleic acid molecule is used as a primer for the RCA, and the 3′ end of the primary immobilizable probe is extended by polymerization in the RCA reaction to generate the RCP.

In some embodiments, the detecting comprises hybridizing a secondary circular probe to the primary barcode sequence in the primary immobilizable probe or hybridizing a secondary circularizable probe to the primary barcode sequence in the primary immobilizable probe and circularizing the secondary circularizable probe to generate a secondary circularized probe. In some embodiments, the detecting further comprises performing rolling circle amplification (RCA) using the secondary circular or circularized probe as a template to generate a rolling circle amplification product (RCP). In some embodiments, the detecting further comprises detecting the RCP at a position in the biological sample or matrix.

In some embodiments, the primary immobilizable probe or product generated therefrom (e.g. RCP) is detected using a detection probe. In some embodiments, the RCP comprises multiple copies of an RCP barcode sequence, and the method comprises hybridizing a detection probe to the RCP barcode sequence and detecting the detection probe or a product thereof, thereby detecting the RCP at the position in the biological sample or matrix. In some embodiments, the detection probe comprises (i) a recognition sequence that hybridizes to a sequence of the RCP and (ii) a reporter sequence for binding directly or indirectly to a reporter oligonucleotide that is associated with a detectable moiety. In some embodiments, detecting the detection probe comprises detecting the detectable moiety of the reporter oligonucleotide bound to the detection probe. In some embodiments, the detection probe is a first detection probe, and the method comprises removing the first detection probe after detecting the first detection probe, hybridizing a second detection probe to the RCP, and detecting the second detection probe. In some embodiments, a sequence of signals is generated, using different detection probes, wherein the sequence of signals corresponds to the target nucleic acid. In some embodiments, the detection probe is directly associated with a detectable moiety. In some embodiments, the detectable moiety is a fluorescent label.

In some embodiments, the detection probe hybridizes to a sequence in a product generated from the primary immobilizable probe other than an RCP. For example, the detection probe can hybridize to a branched hybridization complex, for example as shown in FIG. 3B. Thus, in some embodiments, the detection proceeds generally as described as for methods comprising RCA, but with a different product (e.g. branched hybridization complex).

In some embodiments, the steps of the method provided herein are performed in any suitable order. In some embodiments, the secondary circular or circularizable probe is hybridized to the primary immobilizable probe after the primary immobilizable probe has been attached (e.g. crosslinked) to the biological sample or to the matrix. In some embodiments, the secondary circular or circularizable probe is hybridized to the primary immobilizable probe after the first part of the split hybridization region has been ligated to the second part of the split hybridization region. In some embodiments, the biological sample is a tissue sample and the primary immobilizable probe is attached (e.g. crosslinked) using the attachment moiety to a matrix embedding the tissue sample. In some embodiments, the method comprises digesting the tissue sample before detecting the primary immobilizable probe at a position in the matrix. In some embodiments, the method comprises digesting the tissue sample before detecting the primary immobilizable probe (e.g. by generating an amplification product, such as an RCP or a product of branched DNA amplification) at a position in the matrix. In some embodiments, the method comprises digesting the tissue sample before generating an amplification product to detect the primary immobilizable probe (e.g. by performing RCA of the secondary circular or circularized probe, or by performing branched DNA amplification). In some embodiments, the method comprises digesting the tissue sample before hybridizing the secondary circular or circularizable probe to the primary immobilizable probe. In some embodiments, the biological sample is cleared. In some embodiments, the method comprises clearing the biological sample after attaching (e.g. crosslinking) the primary immobilizable probe to the biological sample or matrix. In some embodiments, the amplification (e.g. RCA or branched DNA amplification) is performed after clearing the biological sample. In some embodiments, the clearing comprises contacting the biological sample with a proteinase. In some embodiments, the method comprises performing one or more wash steps to remove unbound and/or nonspecifically bound probe molecules from the sample.

In some embodiments, the biological sample is cleared. In some embodiments, the clearing is performed in any suitable order in relation to other steps of the method. For example, clearing can be performed before and/or after matrix formation. Different advantages can be associated with clearing at different steps. For example, clearing the biological sample before matrix formation may reduce false positive signals when detecting the target nucleic acid. Clearing the biological sample after matrix formation may lower background signal.

In some embodiments, the target nucleic acid is any suitable target nucleic acid. In some embodiments, the target nucleic acid is DNA, such as cDNA. In some embodiments, the target nucleic acid is RNA, optionally mRNA, an RNA fragment, and/or an RNA fragment that does not comprise a polyA tail. Thus, in some embodiments, the methods provided herein are advantageous for detecting target nucleic acids that may be degraded or fragmented as a consequence of tissue sample preparation, fixation, or other processing steps.

### B. Generation of Products

In some embodiments, provided herein are methods and compositions for analyzing one or more products of an endogenous analyte and/or a labeling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labeling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed.

### (a) Hybridization

In some embodiments, a hybridization product comprising the pairing of substantially complementary or complementary nucleic acid sequences within two different molecules can be analyzed. For example, hybridization of an endogenous analyte or the labeling agent (e.g., reporter oligonucleotide attached thereto) with another endogenous molecule or another labeling agent or a probe can be analyzed. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

### (b) Ligation

In some embodiments, a ligation product of an endogenous analyte and/or a labeling agent is analyzed. In some embodiments, the ligation product is formed between two or more endogenous analytes. In some embodiments, the ligation product is formed between two or more labeling agents. In some embodiments, the ligation product is formed between the first and second part of the split hybridization region of the primary immobilizable probe. In some embodiments, ligating the first part of the split hybridization region to the second part of the split hybridization region of the primary immobilizable probe forms a ligated primary immobilizable probe. In some embodiments, the ligation product is an intramolecular ligation of an endogenous analyte. In some embodiments, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some embodiments, provided herein is a probe capable of DNA-templated ligation (e.g., a primary immobilizable probe comprising two or more nucleic acid molecules to be ligated). In some embodiments, provided herein is a probe or probe set capable of RNA-templated ligation.

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76(14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, circularizable probe or probe set, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions is filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation includes a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### (c) Primer Extension and Amplification

In some embodiments, a primer extension product of an analyte, a labeling agent, a probe or probe set bound to the analyte (e.g., a circularizable probe bound to genomic DNA, mRNA, or cDNA), or a probe or probe set bound to the labeling agent (e.g., a circularizable probe bound to one or more reporter oligonucleotides from the same or different labeling agents) is analyzed.

In some embodiments, a primer extension product is generated by extending a primer than hybridizes to a template. For example, a primer extension product can be generated by extending a primer that is hybridized to a circular template for RCA, thereby generating the primer extension product, which is an RCA product. Thus, in some embodiments, the primer for a primer extension product is an RCA primer. In some embodiments, a primer extension product is generated using the primary immobilizable probe as template. In some embodiments, the primary immobilizable probe includes a splint oligonucleotide, which serves as an RCA primer to generate an RCP using the ligated (and circularized) first and second nucleic acid molecules of the primary immobilizable probe. Because the extended splint oligonucleotide (the RCA product in this embodiment) includes an attachment moiety, the attachment moiety can be used to attach (e.g. immobilize by crosslinking) the RCA product to the matrix. In other embodiments, the primer extension product is generated using the secondary circular or circularized probe as described herein as template.

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers are used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labeling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N⁶-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) are modified to contain functional groups that are used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465, which are herein incorporated by reference in their entireties. In some examples, the scaffold also contains modifications or functional groups that react with or incorporate the modifications or functional groups of the probe or probe set or amplification product. In some examples, the scaffold comprises oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template comprises the target analyte, or a part thereof, where the target analyte is a nucleic acid, or it may be provided or generated as a proxy, or a marker, for the analyte. In some embodiments, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. The RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a probe disclosed herein may be a hybridization complex formed of a cellular nucleic acid in a sample and an exogenously added nucleic acid probe (e.g. the primary immobilizable probe). The exogenously added nucleic acid probe may comprise an overhang that does not hybridize to the cellular nucleic acid but hybridizes to another probe (e.g., the overhang region of the first nucleic acid molecule of the primary immobilizable probe can hybridize to the secondary circular or circularized probe). The exogenously added nucleic acid probe may be optionally ligated to a nucleic acid molecule such as another exogenous nucleic acid molecule (e.g. the split hybridization region of the first and second nucleic acid molecule can be ligated). In other examples, a product comprising a target sequence for a probe disclosed herein (e.g., a detection probe) may be an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a probe disclosed herein (e.g., a reporter oligonucleotide) may be a probe hybridizing to an RCP. The probe may comprise an overhang that does not hybridize to the RCP but hybridizes to another probe.

### III. Crosslinkable Moieties and Interstrand Crosslinking

In some embodiments, a primary immobilizable probe provided herein is crosslinked to the target nucleic acid. Thus, in some embodiments, the primary immobilizable probe comprises a crosslinkable moiety for interstrand crosslinking between the primary immobilizable probe and the target nucleic acid. In some embodiments, the crosslinkable moiety is or is in a photoreactive nucleotide residue. The hybridization region (e.g. split hybridization region) of the primary immobilizable probe may comprise one or more crosslinkable moieties (e.g., photoreactive nucleotide residues). In some embodiments, crosslinking is performed to form an interstrand crosslink between the primary immobilizable probe and the target nucleic acid. In some embodiments, the crosslinking occurs in the split hybridization region of the primary immobilizable probe. In some embodiments, the primary immobilizable probe is crosslinked to the target nucleic acid upon activation by providing a stimulus. In some embodiments, the primary immobilizable probe is crosslinked to the target nucleic acid via the one or more crosslinkable moieties in the split hybridization region. In some aspects, the methods provided herein comprise crosslinking a primary probe or a product thereof (e.g., an RCP) to a biological sample or matrix. In some embodiments, the crosslinkable moiety comprises an activatable chemical group. The crosslinkable moiety or moieties may become photo-activated as described in below, in order to crosslink the primary immobilizable probe to the target nucleic acid in the biological sample.

In some embodiments, activation of the crosslinkable moiety is light driven and performed in aqueous solution. In some embodiments, crosslinking strands of nucleic acid molecules comprise at least one photo-reactive nucleobase. In some embodiments, the crosslinkable moiety is a photo-reactive nucleobase. In some embodiments, the photo-reactive nucleobase is any modified nucleobase that is capable of forming a crosslink with another nucleobase in an opposite hybridized strand in the presence of light. In some embodiments, the photo-reactive nucleobase is a modified pyrimidine or purine nucleobase. In some embodiments, the photo reactive nucleobase comprises a vinyl, acrylate, N-hydroxysuccinimide, amine, carboxylate or thiol chemical group. In some embodiments, the photo-reactive nucleobase comprises a bromo-deoxyuridine. Exemplary photoreactive crosslinkable moieties and photoreactive nucleotides are described, for example, in Elskens and Madder RSC Chem. Biol., 2021, 2, 410-422, the content of which is herein incorporated by reference in its entirety.

In some embodiments, the crosslinkable moiety comprises a reactive chemical group that requires light activation to initiate crosslinking. In some embodiments, the chemical group comprises, for example, an aryl azide, azido-methyl-coumarin, benzophenone, anthraquinone, certain diazo compounds, diazirine, or a psoralen derivative.

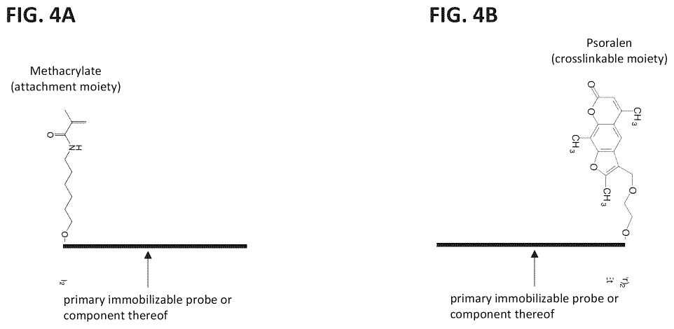

In some embodiments, the crosslinkable moiety comprises a cyanovinylcarbazole moiety. In some embodiments, the crosslinkable moiety comprises a 3-cyanovinylcarbazole (^{CNV}K) nucleoside or 3-cyanovinylcarbazole modified D-threoninol (^{CNV}D). In some embodiments, the crosslinkable moiety comprises 3-cyanovinylcarbazole phosphoramidite. In some embodiments, the crosslinkable moiety comprises a pyranocarbazole. In some embodiments, the crosslinkable moiety comprises a pyranocarbazole (^{PC}X) modified nucleoside or a pyranocarbazole with a D-threoninol instead of a 2′-deoxyribose backbone (^{PCX}D). In some embodiments, the crosslinkable moiety comprises a psoralen (e.g. as shown in FIG. 4B) or a coumarin. In some embodiments, the nucleotide residues comprising crosslinkable moieties have been attached (e.g., by extension with a polymerase or ligation) to a primary immobilizable probe that is hybridized to a target nucleic acid within a sample. In some embodiments, the photoreactive nucleotides have been attached to the primary immobilizable probe via a linker (e.g., a disulfide linker). In some embodiments, the crosslinkable moiety is a photoreactive nucleotide comprising a universal base.

In some embodiments, the crosslinkable moiety is a pyranocarbazole (^{PC}X) modified nucleoside. The ^{PC}X crosslinking base displays high crosslinking efficiency with a thymine (T) base or a cytosine (C) base that is positioned adjacent to the base on the complementary strand and can be directly incorporated into the DNA hybridization domain itself as a base substitution. In some embodiments, a crosslinking reaction is performed using 400 nm wavelength of light and completed within about 10 seconds. In some embodiments, a crosslinking reaction is completed within 0.1, 0.25, 0.5, 1, 5, or 10 seconds. In some embodiments, a crosslinking reaction is completed within 0.5, 1, 5, 10, 20, 30, 40, 50, or 60 minutes. In some embodiments, a crosslinking reaction is completed within 0.5, 1, 2, 3, 4, or 5 minutes. In some embodiments, a crosslinking reaction has negligible effects on bases that neighbor the photoreactive nucleobase. In some embodiments, other photochemical nucleic acid crosslinking agents, including psoralen is used in combination with the photoreactive nucleobases disclosed herein. In some embodiments, a photo-induced crosslink is reversed. In some embodiments, a ^{PC}X crosslink is reversed when exposed to 312305 nm UV light.

In some embodiments, other photochemical nucleic acid crosslinking agents, including psoralen and psoralen derivatives (e.g., psoralen modified nucleosides) are used as crosslinkable moieties. Psoralen and psoralen derivatives can be light-activated with a UV-A of 365 nm. Psoralens react with nearby pyrimidine residues. A variety of nucleosides modified with psoralen or psoralen derivatives may be used. For example, click chemistry using a psoralen azide and a nucleosidic alkyne derivative can be used to generate a variety of photoreactive nucleotides. The psoralen can be connected to the nucleotide via a linker, such as a phosphoramidite. Exemplary psoralen derivatives comprising phosphoramidite include but are not limited to 6-[4′-(Hydroxymethyl)-4,5′, 8-trimethylpsoralen]-hexyl-1-O-(2-cyanoethyl)-(N,N-diisopropyl)-phosphoramidite and 2-[4′-(hydroxymethyl)-4,5′,8-trimethylpsoralen]-ethyl-1-O-(2-cyanoethyl)-(N,N-diisopropyl)-phosphoramidite. In some embodiments, the psoralen or psoralen derivative is conjugated to position 5 of a uridine or pseudouridine (optionally via a linker). In some cases, the psoralen or psoralen derivative is conjugated to the 2′ position of a sugar ring of a uridine or pseudouridine (optionally via a linker). In some embodiments, the psoralen derivative is an amine-reactive derivative, which can be conjugated to an amine-modified nucleotide (e.g., an aminoallyl uridine or pseudouridine nucleotide).

In some embodiments, a psoralen-crosslink (e.g., an interstrand crosslink between the primary immobilizable probe and the target nucleic acid) is reversed when exposed to 254 nm light. In some embodiments, the crosslinkable moiety comprises a C2′ psoralen modification. The crosslinkable moiety can comprise a 5′ psoralen derivative. The structure of two exemplary psoralen-modified oligonucleotides (one 5′ modified nucleoside on the left, and one C2′ modified nucleoside on the right) are shown below:

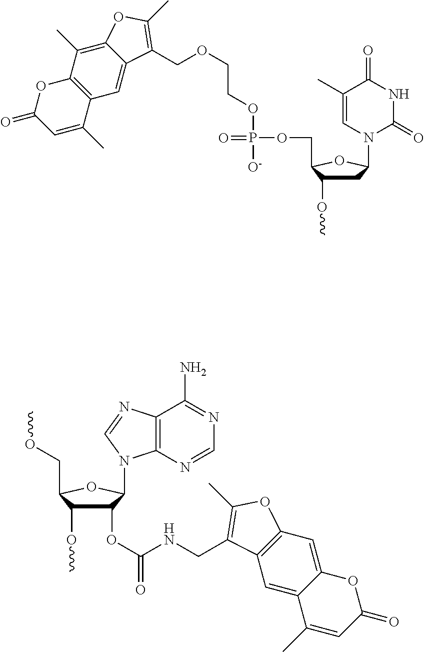

In some embodiments, the crosslinkable moiety is or is linked to a photoactivatable nucleotide, wherein the photoactivatable nucleotide is a universal base such as a pseudouridine modified with a photoreactive moiety (e.g. a psoralen).

In some embodiments, when the primary immobilizable probe comprises ^{CNV}K, rapid photo cross-linking to pyrimidines in the complementary strand (DNA or RNA) is induced at one wavelength and rapid reversal of the cross-link is possible at a second wavelength if desired. Neither wavelength has the potential to cause significant DNA damage and neither interfere with the wavelengths used to excite the fluorophores used during subsequent analysis, such as decoding barcode sequences in situ. Once cross-linked, the UV melting temperature of the duplex may be raised by around 30° C./^{CNV}K moiety relative to the duplex before irradiation and inter-strand crosslinking. The structure of an exemplary 3-cyanovinylcarbazole phosphoramidite is shown below:

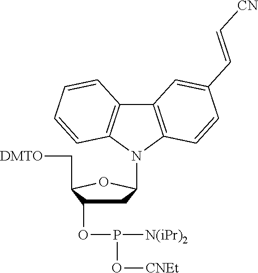

### 5′-O-(4,4′-Dimethoxytrityl)-1′-(3-cyanovinylcarbazol-9-yl)-2′-deoxy-β-D-ribofuranosyl-3′-[(2-cyanoethyl)-(N,N-diisopropyl)]-phosphoramidite

The ^{CNV}K crosslinking base displays high crosslinking efficiency with a thymine (T) base that is positioned adjacent to the base on the opposite hybridized strand in the target nucleic acid (e.g., the complementary strand) (Ultrafast reversible photo-cross-linking reaction: toward in situ DNA manipulation. Org. Lett. 10, 3227-3230 (2008)) and can be directly incorporated into the DNA hybridization domain itself as a base substitution, as shown below in light-directed reaction between a ^{CNV}K base modification and a thymine base to produce a crosslinked nucleic acid.

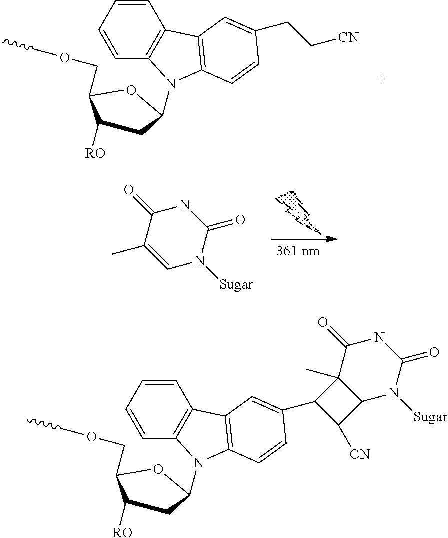

In some embodiments, a crosslinking reaction is performed using 365 nm wavelength of light and can be completed within about 1 second. In some embodiments, a crosslinking reaction is performed using any wavelength of visible or ultraviolet light. In some embodiments, a crosslinking reaction is completed within 0.1, 0.25, 0.5, 1, 5, or 10 seconds. In some embodiments, a crosslinking reaction is completed within 20, 30, 40, 50, or 60 seconds. In some embodiments, the method comprises irradiating the biological sample with UV light, such as a 350-400 nm wavelength of light, for between 10 seconds and 10 minutes, between 10 seconds and 5 minutes, between 10 seconds and 2 minutes, between 10 seconds and 1 minute, between 30 seconds and 1 minute, or between 30 seconds and 5 minutes. In some embodiments, a crosslinking reaction is completed within 0.5, 1, 5, 10, 20, 30, 40, 50, or 60 minutes. In some embodiments, a crosslinking reaction has negligible effects on bases that neighbor the photoreactive nucleobase. In some embodiments, other photochemical nucleic acid crosslinking agents, including psoralen and coumarin are used in combination with the photoreactive nucleobases disclosed herein.

In some embodiments, the crosslinkable moiety comprises a coumarin and the photoactivation comprises irradiating the biological sample using a 350 nm wavelength of light. In some embodiments, the crosslinkable moiety comprises a psoralen and the photoactivation comprises irradiating the biological sample using a 365 nm wavelength of light. In some embodiments, the crosslinkable moiety comprises a ^{CNV}K or ^{CNV}D and the photoactivation comprises irradiating the biological sample using a 365 nm wavelength of light. In some embodiments, the crosslinkable moiety comprises a ^{PC}X or ^{PCX}D and the photoactivation comprises irradiating the biological sample using a 400 nm wavelength of light. In some embodiments, the crosslinkable moiety comprises a diazirine and the photoactivation comprises irradiating the biological sample using a 365 nm wavelength of light. In some embodiments, the crosslinkable moiety comprises a thiouridine and the photoactivation comprises irradiating the biological sample using a 365 nm wavelength of light.

In some embodiments, a photo-induced crosslink is reversed. In some embodiments, a vinylcarbazole (e.g., ^{CNV}K ^{CNV}D, ^{PC}X, or ^{PCX}D) crosslink is reversed when exposed to 305 nm UV light. In some embodiments, a vinylcarbazole (e.g., ^{CNV}K ^{CNV}D, ^{PC}X, or ^{PCX}D) crosslink is reversed when exposed to 312 nm light. In some embodiments, a psoralen crosslink is reversed when exposed to 254 nm light. In some embodiments, a coumarin crosslink is reversed when exposed to 254 nm light.

In some embodiments, the crosslinkable moiety is a photoactivatable nucleotide comprising a coumarin and hybridizes to a thymine (T) base in the complementary strand. In some embodiments, the photoactivatable nucleotide comprises a psoralen and hybridizes to a C, T, or U base in the complementary strand. In some embodiments, the photoactivatable nucleotide comprises a vinylcarbazole and hybridizes to a C, T, or U base in the complementary strand. In some embodiments, the photoactivatable nucleotide comprises a universal or random base.

In some embodiments, the crosslinkable moiety crosslinks to an adenine (A) nucleobase in the strand of the target nucleic acid hybridized to the primary immobilizable probe. In some embodiments, the crosslinkable moiety comprises a psoralen capable of crosslinking to an adenine in the hybridized nucleic acid strand. In some embodiments, the crosslinkable moiety comprises a 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite). In some embodiments, crosslinkable moiety comprises a 5′-Dimethoxytrityl-5-iodo-2′-deoxyUridine,3′-[(2-cyanoethyl)-(N,N-diisopropyl)]-phosphoramidite (5-I-dU-CE phosphoramidite).

The structure of an exemplary psoralen C2 phosphoramidite crosslinkable moiety is shown below:

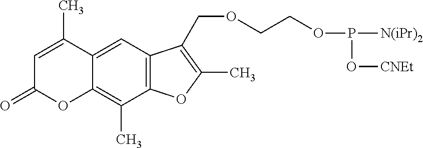

The structure of an exemplary 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite) crosslinkable moiety is shown below:

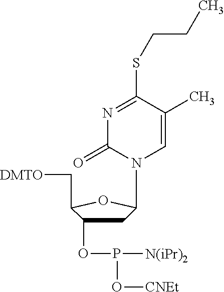

The structure of an exemplary 5′-Dimethoxytrityl-5-iodo-2′-deoxyUridine,3′-[(2-cyanoethyl)-(N,N-diisopropyl)]-phosphoramidite (5-I-dU-CE phosphoramidite) crosslinkable moiety is shown below:

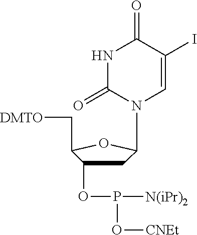

In some embodiments, the target nucleic acid hybridized to the primary immobilizable probe is immobilized within the biological sample or matrix generally at the location of the target nucleic acid hybridized by the primary immobilizable probe, thereby creating a localized nucleic acid concatemer comprising the target nucleic acid. In some cases, the target nucleic acid is covalently linked to the primary immobilizable probe by interstrand crosslinking using the crosslinkable moiety of the primary immobilizable probe. In some embodiments, the target nucleic acid is immobilized within the biological sample or matrix by covalent or noncovalent bonding between the attachment moiety and a molecule in the biological sample or the matrix. In some embodiments, by being immobilized to the biological sample or matrix, the spatial relationship of the original target nucleic acids is maintained. In some embodiments, a primary probe or amplification product thereof is also immobilized in the biological sample or matrix, such as using a primer comprising functional moiety for attachment to the biological sample or matrix (e.g., for generating a rolling circle amplification product) and/or by incorporating one or more crosslinkable moieties into a rolling circle amplification product. In some embodiments, by being immobilized to the target nucleic acid, such as by covalent bonding or cross-linking, the primary immobilizable probe is resistant to movement or dehybridization under mechanical stress. In some embodiments, by being immobilized in the biological sample or matrix, such as by covalent bonding or cross-linking, the rolling circle amplification product is also resistant to movement and/or unraveling under mechanical stress.

The photoreactive nucleotides may be photo-activated by UV light, such as a 350-400 nm wavelength of light, to photo-activate and crosslink the crosslinkable moiety of the hybridized primary immobilizable probe to the target nucleic acid. In some embodiments, the crosslinkable moiety is crosslinked to the complementary strand at a 355 nm wavelength of light. In some embodiments, the purine bases of the target nucleic acid are unreactive to photo-activated crosslinking. In some embodiments, the pyrimidine bases of the complementary strand are reactive to photo-activated crosslinking. In some embodiments, the purine bases of the target nucleic acid are reactive to crosslinking (e.g., to a psoralen, 5-I-dU-CE, 4-Thio-dT-CE, or any other crosslinkable moiety configured to crosslink with nucleobases including adenine).

The photo-activated crosslinking step may be optimized to prevent DNA damage. In some embodiments, the photo-activated crosslinking does not cause significant DNA damage. In some embodiments, the photo-activated crosslinking of the nucleic acid concatemer or the oligonucleotide hybridization region to the complementary strand increases the UV melting temperature of the duplex compared to prior to the crosslinking. In some embodiments, the UV melting temperature is increased by about 30° C. per photoreactive nucleotide in the hybridization region. This increase in melting temperature allows the nucleic acid concatemer to be immobilized to the complementary strand, thereby maintaining spatial fidelity during downstream analyses.

In some embodiments, the photo-activated crosslinking is reversible. In some embodiments, the photo-activated crosslinking is partially reversible. In some embodiments, the photo-activated crosslinking is completely reversible. In some embodiments, the reverse crosslinking comprises exposing the sample to UV light, such as between about 310 nm and 315 nm wavelength of light. In some embodiments, the reverse crosslinking comprises exposing the sample to 312 nm wavelength of light. In some embodiments, the reverse crosslinking comprises about 3 minutes.

In some embodiments, the photo-activated crosslinking and/or immobilization of the target nucleic acid maintains spatial orientation of the target nucleic acid relative to the biological sample or matrix in the presence of denaturing agents. In some embodiments, the method further comprises processing the biological sample comprising the immobilized target nucleic acid. In some embodiments, the processing comprises subjecting the biological sample comprising the crosslinked nucleic acid concatemer to a denaturing condition. In some embodiments, the denaturing condition comprises contacting the biological sample with a denaturing agent and/or heating the biological sample. In some embodiments, the denaturing agent comprises formamide, guanidine, sodium salicylate, dimethyl sulfoxide (DMSO), ethylene carbonate, propylene glycol, or urea. In some embodiments, the denaturing agent comprises formamide. In some embodiments, the denaturing comprises heating the biological sample to disrupt base pairing between the nucleic acid concatemer and the complementary strand. In some embodiments, the biological sample is denatured upon heating above about 80° C. In some embodiments, the biological sample is subjected to repeated cycles of washes that may include denaturing conditions.

### IV. Attachment to the Biological Sample or Matrix

In some embodiments, a primary immobilizable probe according to the present disclosure comprises an attachment moiety that is attached to the biological sample (e.g., to another molecule in the biological sample) or to a matrix. In some embodiments, the attachment moiety is attached to a protein in the biological sample (e.g., by crosslinking). In some embodiments, the attachment moiety is attached to an anchoring moiety in a matrix embedding the biological sample. The attachment can be covalent (e.g., crosslinking) or non-covalent (e.g., interaction between a ligand-ligand binding pair). In some embodiments, the attachment moiety is a crosslinkable moiety, such as any described in Section III.

In some embodiments, the attachment moiety is a reactive group. Exemplary reactive groups for attachment to a biological sample or matrix include, but are not limited to, an amine, a thiol, an azide, an alkyne, a nitrone, an alkene, a tetrazine, a tetrazole, an acrydite or other click reactive group. In some embodiments, the attachment moiety is an acrydite moiety (e.g., as shown in FIG. 4A). In some embodiments, the acrydite is a C6 methacrylate. In some embodiments, the attachment moiety is a methacrylate C6 phosphoramidite.

In some embodiments, the attachment moiety comprises or is an electrophilic group that is capable of interacting with a reactive nucleophilic group present in the biological or matrix to provide a covalent bond. In some embodiments, nucleophilic group is selected from a sulfhydryl, hydroxyl and amino functional group. In some embodiments, the attachment moiety or comprises or is a maleimide, haloacetamide, or NHS ester.

In some embodiments, the attachment moiety comprises or is a nucleophilic group that is capable of interacting with a reactive electrophilic group present in the biological or matrix to provide a covalent bond. In some embodiments, the attachment moiety comprises or is a thiol, phenol, amino, hydrazide, hydroxylamine, hydrazine, thiosemicarbazone, hydrazine carboxylate, or arylhydrazide.

In some embodiments, the attachment moiety comprises or is a click functional group. Suitable click functional groups may include functional groups compatible with a nucleophilic addition reaction, a cyclopropane-tetrazine reaction, a strain-promoted azide-alkyne cycloaddition (SPAAC) reaction, an alkyne hydrothiolation reaction, an alkene hydrothiolation reaction, a strain-promoted alkyne-nitrone cycloaddition (SPANC) reaction, an inverse electron-demand Diels-Alder (IED-DA) reaction, a cyanobenzothiazole condensation reaction, an aldehyde/ketone condensation reaction, and Cu(I)-catalyzed azide-alkyne cycloaddition (CuAAC) reaction. In some embodiments, the attachment moiety comprises or is any functional group involved in click reactions. In some embodiments, such click reactions involve (i) azido and cyclooctynyl; (ii) azido and alkynyl; (iii) tetrazine and dienophile; (iv) thiol and alkynyl; (v) cyano and amino thiol; (vi) nitrone and cyclooctynyl; or (vii) cyclooctynyl and nitrone. It should be recognized that in instances in which the attachment moiety comprises or is a click functional group, the molecule present in the biological or matrix to which it is capable of forming a covalent bond with comprises the complementary click functional group to that of the attachment moiety. For example, in some embodiments, the attachment moiety comprises or is an azide moiety and the molecule present in the biological sample or matrix comprises a complementary alkyne moiety, or vice versa.

In some embodiments, the attachment moiety reacts with a cross-linker. In some embodiments, the attachment moiety is part of a ligand-ligand binding pair. An exemplary attachment moiety includes an amine, amine reactive groups, acrydite, an acrydite modified entity, alkyne, biotin, azide, thiol, and a thiol-modified entity and entities suitable for click chemistry techniques. Biotin, or a derivative thereof, may be used as a matrix attachment moiety when the matrix includes an avidin/streptavidin derivative or an anti-biotin antibody (e.g., a detectably labelled antibody). Similarly, biotin, or a derivative thereof, may be used as a biological sample attachment moiety when the biological sample includes an avidin/streptavidin derivative or an anti-biotin antibody (e.g., a detectably labelled antibody). In some embodiments, a polyacrylamide gel matrix is co-polymerized with acrydite-modified streptavidin. In some instances, biotin or an avidin/streptavidin derivative is attached to the matrix after the matrix is formed. In one example, a polyacrylamide gel matrix is co-polymerized with acrydite-modified streptavidin monomers and biotinylated primary immobilizable probe, using a suitable acrylamide:bis-acrylamide ratio to control the cross-linking density. Digoxigenin may be used as a matrix attachment moiety and subsequently bound by an anti-digoxigenin antibody attached to the matrix. An aminoallyl-dUTP residue may be an attachment moiety incorporated into an primary immobilizable probe and subsequently coupled to an N-hydroxy succinimide which may be incorporated into the matrix. In some embodiments, a Dibenzocyclooctyne (DBCO)-azide attachment moiety is used for matrix attachment. In some embodiments, a DBCO attachment moiety is incorporated into the primary immobilizable probe, and the matrix comprises an azide. In some embodiments, the DBCO is reacted with the azide in a strain promoted alkyne-azide cycloaddition (SPAAC). In some embodiments, an analyte is attached to the matrix using acrydite (e.g., by copolymerization), NHS ester (e.g., coupling of an NHS ester linked to the analyte with an amine on the matrix), DBCO (e.g., coupling a DBCO in the analyte to an azide on the matrix), sulfhydryl (e.g, coupling a sulfhydryl in or associate with the analyte to a maleimide on matrix), amine (e.g., coupling an amine in or associated with the analyte to a carboxyl on the matrix, or coupling a carboxyl in or associated with the analyte to an amine on the matrix). In general, any member of a conjugate pair or reactive pair may be used as an attachment moiety to attach the primary immobilizable probe to a matrix.

Attachment moieties for attachment to a matrix include chemical cross-linking agents. Cross-linking agents typically contain at least two reactive groups that are reactive towards numerous groups, including, but not limited to, sulfhydryls and amines, and create chemical covalent bonds between two or more molecules. Attachment moieties include cross-linking agents such as primary amines, carboxyls, sulfhydryls, carbohydrates and carboxylic acids. Protein molecules have many of these functional groups and therefore proteins and peptides can be readily conjugated using cross-linking agents. Cross-linking agents are commercially available (Thermo Scientific (Rockford, IL)). In the case of crosslinking, the matrix attachment moiety may be attached to modified dNTP or dUTP in the primary immobilizable probe, or to both. Suitable exemplary cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. Such spacer moieties may be functionalized. Such spacer moieties may be chemically stable. Suitable exemplary spacer moieties include polyethylene glycol, carbon spacers, cleavable (e.g., photo-cleavable or chemically cleavable) spacers and other spacers and the like.

In some embodiments, the methods provided herein comprise attaching a primary probe or a product thereof (e.g., an RCP generated from a circular or circularized probe) to the biological sample or matrix. In some embodiments, RCA is performed using a primer comprising a functional moiety for attachment to the biological sample or matrix. The functional moiety can be any of the attachment moieties described above. In some cases, the functional moiety of the primer is orthogonal to the attachment moiety of the primary immobilizable probe. In some embodiments, the method comprises contacting the biological sample or matrix with a nucleotide mixture comprising one or more modified crosslinkable nucleotides for incorporation into the RCP. In some embodiments, the modified crosslinkable nucleotides are functionally orthogonal to the attachment moiety of the primary immobilizable probe. In some cases, the matrix is a multifunctional matrix (e.g., a multifunctional hydrogel), comprising a first anchoring moiety for attachment to the attachment moiety of the primary immobilizable probe, and a second anchoring moiety for attachment to the functional moiety of the RCA primer and/or the crosslinkable nucleotides incorporated into the RCP. For example, the first anchoring moiety can be an acrydite moiety for covalent attachment of a methacrylate attachment moiety of the primary immobilizable probe, and the second anchoring moiety can be a methylsulphone moiety for attachment to a thiolated functional moiety in the RCA primer and/or alpha-thiol nucleotides incorporated into the RCP. In some cases, the attachment moiety in the primary immobilizable probe and the functional moiety in the primer and/or crosslinkable nucleotides can be the same.

In some embodiments, an RCP is generated in the biological sample or matrix, wherein the RCP comprises modified nucleotide residues having functional linkage groups for tethering to a matrix, such as acrylamide or click-reactive groups, enabling the products of amplification to be spatially immobilized via covalent gel linkages. According to one aspect, the functional linkages can be incorporated during amplification using nucleotide analogs, including amino-allyl dUTP, 5-TCOPEG4-dUTP, C8-Alkyne-dUTP, 5-Azidomethyl-dUTP, 5-Vinyl-dUTP, 5-Ethynyl dUTP, or a combination thereof. According to a separate aspect, for amplification methods using one or more primers, one or more of the primers can comprise a functional linkage group for tethering to a matrix, e.g., solid-state.

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

Matrix forming materials include polyacrylamide, cellulose, alginate, polyamide, cross-linked agarose, cross-linked dextran or cross-linked polyethylene glycol. The matrix forming materials can form a matrix by polymerization and/or crosslinking of the matrix forming materials using methods specific for the matrix forming materials and methods, reagents and conditions.

In some embodiments, a matrix-forming material is introduced into a cell, for example according to the following procedure. The cells are fixed with formaldehyde and then immersed in ethanol to disrupt the lipid membrane. The matrix forming reagents are added to the sample and are allowed to permeate throughout the cell. A polymerization inducing catalyst, UV or functional cross-linkers are then added to allow the formation of a gel matrix. The un-incorporated material is washed out and any remaining functionally reactive group is quenched. Exemplary cells include any cell, human or otherwise, including diseased cells or healthy cells. Certain cells include human cells, non-human cells, human stem cells, mouse stem cells, primary cell lines, immortalized cell lines, primary and immortalized fibroblasts, HeLa cells and neurons.

In some embodiments, a matrix-forming material is used to encapsulate a biological sample, such as a tissue sample, for example as follows. Formalin-fixed embedded tissues on glass slides are incubated with xylene and washed using ethanol to remove the embedding wax. They are then treated with Proteinase K to permeabilized the tissue. A polymerization inducing catalyst, UV or functional cross-linkers are then added to allow the formation of a gel matrix. The un-incorporated material is washed out and any remaining functionally reactive group is quenched.

In some embodiments, the matrix-forming material forms a three dimensional matrix including a plurality of analytes, target nucleic acid molecules, and/or primary immobilizable probes hybridized thereto while maintaining the spatial relationship of the analytes, target nucleic acid molecules, and/or primary immobilizable probes. In some embodiments, the primary immobilizable probes are immobilized within the matrix by covalent attachment or through ligand-ligand interaction to the matrix. In some embodiments, the primary immobilizable probe hybridized to the target nucleic acid is used to attach the target nucleic acid covalently or non-covalently to the matrix.

In some embodiments, the matrix is sufficiently optically transparent or otherwise has optical properties suitable for deep three dimensional imaging for high throughput information readout, such as for detection of the RCA product using labeled probes (e.g., fluorescently labeled probes).

In some embodiments, the matrix is porous thereby allowing the introduction of reagents (e.g., primary probes, intermediate probes, and/or detectably labeled probes) into the matrix at the site of a target nucleic acid molecule immobilized in the matrix. Additional control over the molecular sieve size and density can be achieved by adding additional cross-linkers such as functionalized polyethylene glycols. In some embodiments, the target nucleic acid molecules are readily accessed by probes, enzymes, and other reagents with rapid kinetics. Porosity can result from polymerization and/or crosslinking of molecules used to make the matrix material. In some aspects, the diffusion property within the gel matrix is largely a function of the pore size. The molecular sieve size can be chosen to allow for rapid diffusion of enzymes, oligonucleotides, formamide, and other buffers used for amplification and detection (>50-nm). The molecular sieve size can also be chosen so that large DNA or RNA amplicons do not readily diffuse within the matrix (<500-nm). The porosity can be controlled by changing the cross-linking density, the chain lengths, and/or the percentage of co-polymerized branching monomers.

In some embodiments, the biological sample is immobilized in the hydrogel via cross-linking of the polymer material that forms the hydrogel. Cross-linking can be performed chemically and/or photochemically, or alternatively by any other hydrogel-formation.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

In some embodiments, a matrix is formed using photopolymerization. In some aspects, photopolymerization involves the use of photons to initiate a polymerization reaction. The photopolymerization reaction can be initiated by a single-photon or a multiphoton excitation system as described elsewhere herein. Light can be manipulated to form specific 2D or 3D patterns and be used to initiate the photopolymerization reaction. In some embodiments, a particular shape or pattern for the 3D matrix is constructed, for example such that the matrix is generated in one part of the cell or cell derivative but not generated in another part of the cell or cell derivative. Light and patterns of light can be generated by spatial light modulators, such as a digital spatial light modulator. The spatial light modulators can employ a transmissive liquid crystal, reflective liquid crystal on silicon (LCOS), digital light processing, a digital micromirror device (DMD), or a combination thereof.

The fixative/hydrogel composition can comprise any hydrogel subunits, such as, but not limited to, poly(ethylene glycol) and derivatives thereof (e.g., PEG-diacrylate (PEG-DA), PEG-RGD), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose and the like. Agents such as hydrophilic nanoparticles, e.g., poly-lactic acid (PLA), poly-glycolic acid (PLG), poly(lactic-co-glycolic acid) (PLGA), polystyrene, poly(dimethylsiloxane) (PDMS), etc. can be used to improve the permeability of the hydrogel while maintaining patternability. Materials such as block copolymers of PEG, degradable PEO, poly (lactic acid) (PLA), and other similar materials can be used to add specific properties to the hydrogel. Crosslinkers (e.g., bis-acrylamide, diazirine, etc.) and initiators (e.g., azobisisobutyronitrile (AIBN), riboflavin, L-arginine, etc.) can be included to promote covalent bonding between interacting macromolecules in later polymerization.

Examples of suitable attachment moieties or functional moieties include electrophiles or nucleophiles that can form a covalent linkage by reaction with a corresponding nucleophile or electrophile, respectively, on the substrate of interest. Non-limiting examples of suitable electrophilic reactive groups can include, for example, esters including activated esters (such as, for example, succinimidyl esters), amides, acrylamides, acridines, acyl azides, acyl halides, acyl nitriles, aldehydes, ketones, alkyl halides, alkyl sulfonates, anhydrides, aryl halides, aziridines, boronates, carbodiimides, diazoalkanes, epoxides, haloacetamides, haloplatinates, halotriazines, imido esters, isocyanates, isothiocyanates, maleimides, phosphoramidites, silyl halides, sulfonate esters, sulfonyl halides, and the like. Non-limiting examples of suitable nucleophilic reactive groups can include, for example, amines, anilines, thiols, alcohols, phenols, hydrazines, hydroxylamines, carboxylic acids, glycols, heterocycles, and the like. Further non-limiting examples of functional moieties include acrydite, biotin, alkyne, and amine groups. In some embodiments, the primary immobilizable probe or other nucleic acids (such as a primer for RCA) comprise an attachment/functional moiety. Functional moieties can be incorporated into oligonucleotides by, for example, incorporation during chemical oligonucleotide synthesis, or chemical conjugation to an oligonucleotide.

In some embodiments, after attachment of the primary immobilizable probe to the matrix, the matrix is partially or substantially cleared of certain species or classes of biomolecules, such as lipids and proteins, as by use of detergent and/or protease reagents. According to some aspects of the present disclosure, the sample can be cleared using a detergent solution, such as Triton-X or SDS. The detergent can interact with the molecules allowing the molecules to be washed out or removed. Other non-limiting examples of detergents include Triton X-100, Triton X-114, Tween-20, Tween 80, saponin, CHAPS, and NP-40. In some embodiments, the sample is cleared using a protease reaction, such as Proteinase K. The protease can cleave or digest proteins such that the fragments or amino acids can be removed. In some embodiments, the extracellular matrix is substantially cleared using one or more specific or nonspecific proteases. Other non-limiting examples of protease include trypsin, chymotrypsin, papain, thrombin, and pepsin.

In some embodiments, the biological sample or the matrix is immobilized onto a solid substrate, such as glass or plastic, facilitating handling and reagent exchange. In some embodiments, a matrix is affixed to a glass slide via oxysilane-functionalization with acrylamide- or free-radical-polymerizing groups, such as methacryloxypropyltrimethoxysilane. The 3D matrix can be free-floating or otherwise not attached to a solid substrate.

Additional methods and aspects of hydrogel embedding of biological samples are described for example in Chen et al., Science 347(6221):543-548, 2015, the content of which is herein incorporated by reference in its entirety.

### V. Detection and Analysis

In some aspects, the provided methods involve analyzing, e.g., detecting or determining, one or more sequences present in the probes or probe sets or products thereof (e.g., rolling circle amplification products thereof). In some embodiments, the detecting is performed at one or more locations in the biological sample and/or matrix (e.g. matrix embedding the biological sample). In some embodiments, the locations are the locations of target nucleic acids, such as RNA transcripts or fragments thereof in the biological sample. In some embodiments, the locations are the locations at which the probes hybridize to the target nucleic acids in the biological sample, and are optionally ligated and amplified by rolling circle amplification. In some embodiments, the primary barcode sequence of the primary immobilizable probe is detected as described herein, e.g., using hybridized probes and/or amplification methods.

In some embodiments, detecting the one or more sequences present in the probes in the biological sample is performed. In some embodiments, the detected sequences are compared to an expected set of detected sequences. In some embodiments, the one or more sequences are one or more barcode sequences or complements thereof.

In some embodiments, the detecting comprises a plurality of repeated cycles of hybridization and removal of probes (e.g., detectably labeled probes, or intermediate probes that bind to detectably labeled probes). In some embodiments, the hybridization is to the primary probe hybridized to the target nucleic acid, or to a product (such as a rolling circle amplification product) generated from the probe hybridized to the target nucleic acid.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization assays, or sequencing by hybridization).

In some embodiments, the detecting comprises binding an intermediate probe directly or indirectly to the primary probe, binding a detectably labeled probe directly or indirectly to a detection region of the intermediate probe, and detecting a signal associated with the detectably labeled probe. In some embodiments, the method comprises detecting a rolling circle amplification product (RCP) generated using a circular or circularized primary probe as a template. In some embodiments, the method comprises detecting a rolling circle amplification product (RCP) generated using a circular or circularized probe or probe that binds to a primary probe as a template. In some embodiments, detecting the RCP comprises binding an intermediate probe directly or indirectly to the RCP, binding a detectably labeled probe directly or indirectly to a detection region of the intermediate probe, and detecting a signal associated with the detectably labeled probe. In some embodiments, the method comprises performing one or more wash steps to remove unbound and/or nonspecifically bound intermediate probe molecules from the primary probes or the products of the primary probes.

In some embodiments, the detecting comprises: detecting signals associated with detectably labeled probes that are hybridized to barcode regions or complements thereof in the primary probe or a product thereof (e.g., an RCP); and/or detecting signals associated with detectably labeled probes that are hybridized to intermediate probes which are in turn hybridized to the barcode regions or complements thereof. In some embodiments, the detectably labeled probes are fluorescently labeled.

In some embodiments, the methods comprise detecting the sequence in all or a portion of a primary probe or an RCP, or detecting a sequence of the primary probe or RCP, such as one or more barcode sequences present in the primary probe (e.g. the primary barcode sequence provided herein) or RCP (e.g. the RCP barcode sequence provided herein). In some embodiments, the sequence of the RCP, or barcode thereof, is indicative of a sequence of the target nucleic acid to which the RCP is hybridized. In some embodiments, the analysis and/or sequence determination comprises detecting a sequence in all or a portion of the nucleic acid concatemer and/or in situ hybridization to the RCP. In some embodiments, the detection step involves sequencing by hybridization, sequencing by ligation, sequencing by synthesis, sequencing by binding, and/or fluorescent in situ sequencing (FISSEQ), and/or hybridization-based in situ sequencing. In some embodiments, the detection step is by sequential fluorescent in situ hybridization (e.g., for combinatorial decoding of the barcode sequence or complement thereof).

In some embodiments, the detection or determination comprises hybridizing to the probe directly or indirectly a detection oligonucleotide labeled with a fluorophore, an isotope, a mass tag, or a combination thereof. In some embodiments, the detection or determination comprises imaging the probe hybridized to the target nucleic acid (e.g., imaging one or more detectably labeled probes hybridized thereto). In some embodiments, the target nucleic acid is an mRNA (or fragment thereof) in a tissue sample, and the detection or determination is performed when the target nucleic acid and/or the amplification product is in situ in the tissue sample. In some embodiments, the target nucleic acid is an amplification product (e.g., a rolling circle amplification product).

In some instances, the disclosed methods may comprise the use of a branched DNA (bDNA) amplification approach to amplify signals. In branched DNA (bDNA) amplification, primary and secondary amplifier oligonucleotides, each containing multiple replicate binding sites, are assembled on, e.g., individual smFISH probes to form a branched structure which binds multiple copies of a fluorescently labeled probe (Xia, et al. (2019), “Multiplexed Detection of RNA Using MERFISH and Branched DNA Amplification”, Scientific Reports 9:7721). The degree of amplification in bDNA amplification is controlled by the design of the amplification reaction, i.e., the assembled bDNA structures cannot grow indefinitely even in the presence of excess reagents, which may be used to control spot size or limit the variability in brightness from molecule to molecule (Xia, et al. (2019), ibid.). In some embodiments, the bDNA amplification is carried out to detect the primary immobilizable probe, for example via binding and/or hybridization of the branched structure to the primary barcode sequence. Embodiments of methods and compositions for branched signal amplification include those described in U.S. Pat. Pub. No. US 2020/0399689 incorporated by reference herein.

In some instances, the disclosed methods may comprise the use of a hybridization chain reaction (HCR) approach to amplify signals. For example, in some embodiments, HCR is used to detect the primary immobilizable probe. For example, in some embodiments, the primary barcode sequence of the primary immobilizable probe (or a corresponding sequence on a product thereof) can serve as an initiator for HCR, or can be hybridized by an oligonucleotide comprising an initiator for HCR. Thus, in some embodiments, the product of the HCR is hybridized and/or bound directly or indirectly to the primary barcode sequence of the primary immobilizable probe. HCR is an enzyme-free nucleic acid amplification based on a triggered chain of hybridization of nucleic acid molecules starting from HCR monomers, which hybridize to one another to form a nicked double-stranded nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. No. 7,632,641 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401), the content of each which is herein incorporated by reference in its entirety. HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction can be carried out with more than two species or types of HCR monomers. For example, a system involving three HCR monomers can be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers can readily be conceived.

In some embodiments of HCR, two fluorescently-labeled metastable hairpin oligonucleotides self-assemble into long fluorescent polymers starting from an initiator sequence present on each probe molecule (Xia, et al. (2019), ibid.). The degree of amplification achieved through HCR can be tuned by changing the hybridization or polymerization times, and can be adjusted to achieve highly amplified signals (which may, however, increase the size of the fluorescent spots generated and/or lead to variable degrees of amplification for different copies of the same target molecule).

In some embodiments, similar to HCR reactions that use hairpin monomers, linear oligo hybridization chain reaction (LO-HCR) are also used for signal amplification. In some embodiments, an initiator is contacted with a plurality of LO-HCR monomers of at least a first and a second species to generate a polymeric LO-HCR product that can be detected. In some embodiments, the first species and/or the second species does not comprise a hairpin structure. In some embodiments, the plurality of LO-HCR monomers does not comprise a metastable secondary structure. In some embodiments, the LO-HCR polymer does not comprise a branched structure. Similar to HCR reactions, LO-HCR reactions can be used to detect the primary immobilizable probe, e.g. via the primary barcode sequence, as described for HCR above. Exemplary methods and compositions for LO-HCR are described in US 2021/0198723, incorporated herein by reference in its entirety.

In some embodiments, the detection comprises signal amplification by performing a primer exchange reaction (PER), for instance, on a ligated probe (e.g., a primary immobilizable probe disclosed herein, such as shown in FIG. 1A) or an RCP. In various embodiments, a primer with a domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a sample comprising primary barcode sequences in hybridized and ligated primary immobilizable probes as described herein. In various embodiments, the primary immobilizable probes or products may be contacted with a plurality of concatemer primers and a plurality of labeled probes. See e.g., U.S. Pat. Pub. No. US20190106733, which is incorporated herein by reference in its entirety, for exemplary molecules and PER reaction components.

In various embodiments, a plurality of concatemer primers is contacted with a sample. In various embodiments, an assembly includes a plurality of concatemer primers, a plurality of labeled probes, and a sample including nucleic acids. In various embodiments, each the plurality of concatemer primers each includes domain 1, 2, 3, etc. In various embodiments, each the plurality of labeled probes each include domain 1′, 2′, 3′, etc., with each corresponding domain 1′, 2′, 3′ being complementary to domain 1, 2, 3, etc., respectively. In various embodiments, the assembly includes the plurality of concatemer primers, which are capable of hybridizing to target nucleic acid sequences in the sample. Described herein is a method using the aforementioned assembly, including contacting the sample including target nucleic acids with the plurality of concatemer primers, then contacting the sample and plurality of concatemer primers with the plurality of labeled probes, thereby labeling the target nucleic acid sequences with a plurality of labeled probes. See e.g., Kishi et al., SABER amplifies FISH: enhanced multiplexed imaging of RNA and DNA in cells and tissues, Nat. Methods. (2019), Saka et al., Immuno-SABER enables highly multiplexed and amplified protein imaging in tissues. Nat. Biotechnol. (2019), and U.S. Pat. Pub. No. 2021/0147902, each of which is fully incorporated by reference herein.

In some aspects, the provided methods comprise imaging a detectably labeled probe bound directly or indirectly to the primary probe or product thereof and detecting the detectable label. In some embodiments, the detectably labeled probe comprises a detectable label that can be measured and quantitated. The label or detectable label can comprise a directly or indirectly detectable moiety, e.g., any fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. Background fluorescence can include autofluorescence (that can arise from a variety of sources, including aldehyde fixation, extracellular matrix components, red blood cells, lipofuscin, and the like), as opposed to the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. In some embodiments, a fluorescent label comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a oligonucleotide sequence is indirectly labeled, especially with a hapten that is then bound by a capture agent, e.g., as disclosed in U.S. Pat. Nos. 5,344,757, 5,702,888, 5,354,657, 5,198,537 and 4,849,336, and 5,073,562, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe (either directly or via a reporter oligonucleotide hybridized directly or indirectly thereto). In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, the assay comprises in situ sequencing. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932, all of which are herein incorporated by reference in their entireties. Exemplary techniques for in situ sequencing or in in situ sequence detection comprise, but are not limited to, STARmap (described for example in Wang et al., (2018) Science, 361(6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48(19):e112, and FISSEQ (described for example in US 2019/0032121, the content is herein incorporated by reference in its entirety).

In some embodiments, analyzing, e.g., detecting or determining, one or more sequences present in the biological sample is performed using a base-by-base sequencing method, e.g., sequencing-by-synthesis (SBS), sequencing-by-avidity (SBA) or sequencing-by-binding (SBB). In some embodiments, the biological sample is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

Generally in sequencing-by-synthesis methods, a first population of detectably labeled nucleotides (e.g., dNTPs) are introduced to contact a template nucleotide (e.g., a barcode sequence in the RCP) hybridized to a sequencing primer, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced, but in order for a second detectably labeled nucleotide to incorporate into the extended sequencing primer, nucleotides in the first population of nucleotides that have not incorporated into a sequencing primer are generally removed (e.g., by washing), and a second population of detectably labeled nucleotides are introduced into the reaction. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in some embodiments, cycles of introducing and removing detectably labeled nucleotides are performed.

In some embodiments, the base-by-base sequencing comprises using a polymerase that is fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

In some embodiments, sequencing is performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing is performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, sequencing is performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

In some embodiments, detection of the barcode sequences is performed by sequential hybridization of probes to the barcode sequences or complements thereof and detecting complexes formed by the probes and barcode sequences or complements thereof. In some cases, each barcode sequence or complement thereof is assigned a sequence of signal codes that identifies the barcode sequence or complement thereof (e.g., a temporal signal signature or code that identifies the analyte), and detecting the barcode sequences or complements thereof can comprise decoding the barcode sequences of complements thereof by detecting the corresponding sequences of signal codes detected from sequential hybridization, detection, and removal of sequential pools of intermediate probes (e.g., the detection probes described herein) and detectably labeled probes (e.g., reporter oligonucleotides as described herein). In some embodiments, a detection probe disclosed herein hybridizes to an RCP. In some embodiments, a reporter oligonucleotide binds directly or indirectly to a detection probe and can be associated with or comprise a detectable moiety or detectable sequence (e.g., a sequence that hybridizes to a detectably labeled oligonucleotide). In some cases, the sequences of signal codes can be fluorophore sequences assigned to the corresponding barcode sequences or complements thereof. In some embodiments, the detectably labeled probes are fluorescently labeled. In some embodiments, the barcode sequence or complement thereof is performed by sequential probe hybridization as described in US 2021/0340618, the content of which is herein incorporated by reference in its entirety.

In some embodiments, the detecting step comprises contacting the biological sample with one or more detectably labeled probes that directly or indirectly hybridize to the barcode sequences or complements thereof (e.g., in amplification products generated using the probes or probe sets), and dehybridizing the one or more detectably labeled probes. In some embodiments, the contacting and dehybridizing steps are repeated with the one or more detectably labeled probes and/or one or more other detectably labeled probes that directly or indirectly hybridize to the barcode sequences or complements thereof. In some aspects, the method comprises sequential hybridization of detectably labeled probes to create a spatiotemporal signal signature or code that identifies the analyte.

In some embodiments, the detecting step comprises contacting the biological sample with one or more first detectably labeled probes that directly hybridize to the plurality of probes or probe sets. In some instances, the detecting step comprises contacting the biological sample with one or more first detectably labeled probes that indirectly hybridize to the plurality of probes or probe sets. In some embodiments, the detecting step comprises contacting the biological sample with one or more first detectably labeled probes that directly or indirectly hybridize to the plurality of probes or probe sets.

In any of the embodiments herein, the detecting step comprises contacting the biological sample with one or more intermediate probes (e.g., the detection probes described herein) that directly or indirectly hybridize to the barcode sequences or complements thereof (e.g., of the plurality of probes or probe sets or rolling circle amplification product generated using the plurality of probes or probe sets), wherein the one or more intermediate probes are detectable using one or more detectably labeled probes. In some embodiments, the detecting step further comprises dehybridizing the one or more intermediate probes and/or the one or more detectably labeled probes from the barcode sequences or complements thereof (e.g., of the plurality of probes or probe sets or rolling circle amplification product generated using the plurality of probes or probe sets). In some embodiments, the contacting and dehybridizing steps are repeated with the one or more intermediate probes, the one or more detectably labeled probes, one or more other intermediate probes, and/or one or more other detectably labeled probes. In some cases, the repeated contacting, detection and dehybridizing steps allows detection of barcode sequences or complements thereof and identification of the corresponding sequences of signal codes (e.g., fluorophore sequences assigned to the corresponding barcode sequences or complements thereof).

In some embodiments, sequencing is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated by reference in their entireties.

In some embodiments, nucleic acid hybridization is used for sequencing. Such methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties.

In some embodiments, real-time monitoring of DNA polymerase activity is used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some embodiments, the analysis and/or sequence determination involves washing to remove unbound polynucleotides, thereafter revealing a fluorescent product for imaging.

### VI. Samples, Analytes, and Target Sequences

### A. Samples

A sample disclosed herein can be or be derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample comprises cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, a substrate herein is any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate is coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some embodiments, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample is prepared using formalin-fixation and paraffin-embedding (FFPE), which are established methods. In some embodiments, cell suspensions and other non-tissue samples are prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or circularizable probe. In some embodiments, one or more post-fixing step is performed after a hybridization complex comprising a probe and a target is formed in a sample. In some embodiments, one or more post-fixing step is performed prior to a ligation reaction disclosed herein.

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

In some embodiments, a biological sample is permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample is permeabilized by any suitable methods. For example, one or more lysis reagents can be added to the sample. In some embodiments, processing of the biological sample, such as de-waxing, allows the biological sample to become permeabilized. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ii) Embedding

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) are embedded in a 3D matrix. In some embodiments, a 3D matrix comprises a network of natural molecules and/or synthetic molecules that are chemically and/or enzymatically linked, e.g., by crosslinking. In some embodiments, a 3D matrix comprises a synthetic polymer. In some embodiments, a 3D matrix comprises a hydrogel.

In some aspects, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof are modified to contain functional groups that are used as an anchoring site to attach the polynucleotide probes and/or amplification product to a polymer matrix. In some embodiments, a modified probe comprising oligo dT is used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel includes hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference. In some embodiments, the hydrogel forms the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible. In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some embodiments, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a biological sample embedded in a matrix (e.g., a hydrogel) is isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(iii) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain is specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample are segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain includes but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample is stained with haematoxylin and eosin (H & E).

The sample can be stained using hematoxylin and eosin (H & E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample is stained using Romanowsky stain, including Wright's stain, Jenner's stain, Can-Grunwald stain, Leishman stain, and Giemsa stain.

In some embodiments, biological samples are destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### B. Analytes

A biological sample may comprise one or a plurality of analytes of interest. Methods for performing multiplexed assays to analyze two or more different analytes in a single biological sample are provided. The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected. In some embodiments, an analyte is a nucleic acid, such as a target nucleic acid as described herein.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

In some embodiments, an analyte herein is endogenous to a biological sample and can include nucleic acid analytes and non-nucleic acid analytes. For example, in some embodiments, the analyte is a target nucleic acid as described herein. In some embodiments, the target nucleic acid is endogenous to the biological sample. Methods and compositions disclosed herein can be used to analyze nucleic acid analytes (e.g., using a nucleic acid probe or probe set that directly or indirectly binds (e.g. hybridizes) to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte is an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte is a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

### (ii) Labeling Agents

In some embodiments, provided herein are methods and compositions for analyzing endogenous analytes (e.g., RNA, ssDNA, cell surface or intracellular proteins, and/or metabolites) in a sample using one or more labeling agents. In some embodiments, an analyte labeling agent includes an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes a barcode that is associated with or otherwise identifies the analyte binding moiety. In some embodiments, by identifying an analyte binding moiety by identifying its associated analyte binding moiety barcode, the analyte to which the analyte binding moiety binds is also identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety includes any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample are subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### C. Barcode Sequences

In some embodiments, an analyte described herein is associated with one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides. In some embodiments, the primary immobilizable probe comprises a barcode (e.g. the primary barcode sequence). In some embodiments, a product generated from the primary immobilizable probe comprises a barcode (e.g. the RCP barcode sequence).

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In any of the preceding embodiments, the methods provided herein can include analyzing the barcodes by sequential hybridization and detection with a plurality of labelled probes (e.g., detection oligos).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub 20210164039, which are hereby incorporated by reference in their entirety.

### VII. Compositions and Kits

In some aspects, provided herein are compositions comprising any of the nucleic acids and/or probes described herein, such as any of the primary immobilizable probes or parts thereof, any of the secondary circular or circularizable probes, any of the detection probes, any of the reporter oligonucleotides, or any of the target nucleic acids provided herein. In some embodiments, the compositions comprise any of the circular primary immobilizable probes described herein. In some embodiments, the circular primary immobilizable probes of the compositions include a splint oligonucleotide. Also provided herein are kits, for analyzing an analyte in a biological sample according to any of the methods described herein.

In some embodiments, provided herein is a kit comprising a primary immobilizable probe, wherein: the primary immobilizable probe comprises a primary barcode sequence, an attachment moiety, and a split hybridization region comprising a first part and a second part, wherein: the first part of the split hybridization region comprises a first nucleotide sequence complementary to a first portion of a region of interest in a target nucleic acid, and the second part of the split hybridization region comprises a second nucleotide sequence complementary to a second portion of the region of interest in the target nucleic acid; the first part and second part are capable of being ligated using the region of interest; and the attachment moiety is capable of being attached (e.g. crosslinked) to a biological sample or matrix embedding the biological sample.

In some embodiments, provided herein is a kit comprising a primary immobilizable probe, wherein: the primary immobilizable probe comprises: (1) a first nucleic acid molecule comprising (i) a first part of a split hybridization region and (ii) an overhang region comprising a primary barcode sequence; and (2) a second nucleic acid molecule comprising (i) a second part of the split hybridization region and (ii) an attachment moiety, wherein: the first part of the split hybridization region comprises a first nucleotide sequence complementary to a first portion of a region of interest in a target nucleic acid, and the second part of the split hybridization region comprises a second nucleotide sequence complementary to a second portion of the region of interest in the target nucleic acid; the first part and second part are capable of being ligated using the region of interest; and the attachment moiety is capable of being attached to a biological sample or matrix embedding the biological sample.

The various components of the kit may be present in separate containers or certain compatible components may be pre-combined into a single container. In some embodiments, the kits further contain instructions for using the components of the kit to practice the provided methods.

In some embodiments, the kits contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer, ligation buffer, and/or reagents for attaching the. In some embodiments, the kits contain reagents for detection and/or sequencing, such as barcode detection probes or detectable labels. In some embodiments, the kits contain other components, for example nucleic acid primers (e.g. RCA primers).

### VIII. Opto-Fluidic Instruments for Analysis of Biological Samples

Provided herein is an instrument having integrated optics and fluidics modules (an “opto-fluidic instrument” or “opto-fluidic system”) for detecting target molecules (e.g., nucleic acids, proteins, antibodies, etc.) in biological samples (e.g., one or more cells or a tissue sample) as described herein. In an opto-fluidic instrument, the fluidics module is configured to deliver one or more reagents (e.g., detectably labeled probes) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more probing cycles (e.g., as described in Section V). In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules (e.g., as described in Section II) in their naturally occurring place (i.e., in situ) within the biological sample. For example, an opto-fluidic instrument may be an in-situ analysis system used to analyze a biological sample and detect target molecules including but not limited to DNA, RNA, proteins, antibodies, and/or the like.

It is to be noted that, although the above discussion relates to an opto-fluidic instrument that can be used for in situ target molecule detection via probe hybridization, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

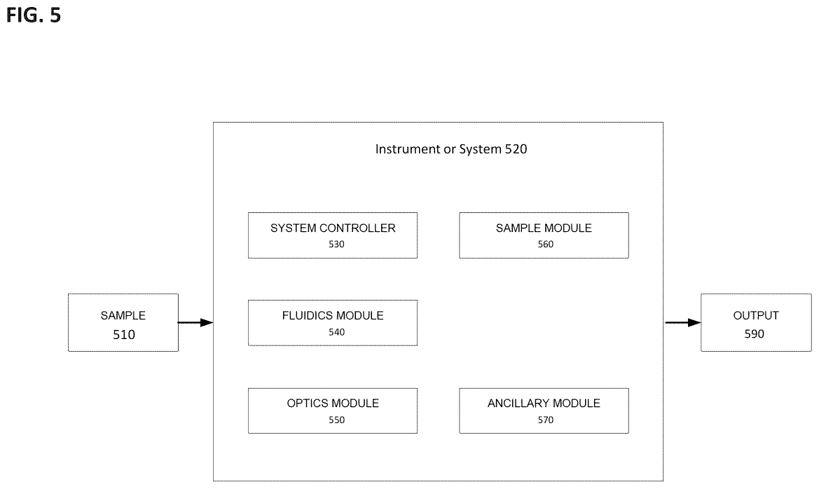

FIG. 5 shows an example workflow of analysis of a biological sample 510 (e.g., cell or tissue sample) using an opto-fluidic instrument 520, according to various embodiments. In various embodiments, the sample 510 can be a biological sample (e.g., a tissue) that includes molecules such as DNA, RNA, proteins, antibodies, etc. For example, the sample 510 can be a sectioned tissue that is treated to access the RNA thereof for labeling with probes described herein (e.g., in Section II). Ligation of the probes may generate a circular probe which can be enzymatically amplified and bound with detectably labeled probes, which can create bright signal that is convenient to image and has a high signal-to-noise ratio.

In various embodiments, the sample 510 may be placed in the opto-fluidic instrument 520 for analysis and detection of the molecules in the sample 510. In various embodiments, the opto-fluidic instrument 520 can be a system configured to facilitate the experimental conditions conducive for the detection of the target molecules. For example, the opto-fluidic instrument 520 can include a fluidics module 540, an optics module 550, a sample module 560, and an ancillary module 570, and these modules may be operated by a system controller 530 to create the experimental conditions for the probing of the molecules in the sample 510 by selected probes (e.g., circularizable DNA probes), as well as to facilitate the imaging of the probed sample (e.g., by an imaging system of the optics module 550). In various embodiments, the various modules of the opto-fluidic instrument 520 may be separate components in communication with each other, or at least some of them may be integrated together.

In various embodiments, the sample module 560 may be configured to receive the sample 510 into the opto-fluidic instrument 520. For instance, the sample module 560 may include a sample interface module (SIM) that is configured to receive a sample device (e.g., cassette) onto which the sample 510 can be deposited. That is, the sample 510 may be placed in the opto-fluidic instrument 520 by depositing the sample 510 (e.g., the sectioned tissue) on a sample device that is then inserted into the SIM of the sample module 560. In some instances, the sample module 560 may also include an X-Y stage onto which the SIM is mounted. The X-Y stage may be configured to move the SIM mounted thereon (e.g., and as such the sample device containing the sample 510 inserted therein) in perpendicular directions along the two-dimensional (2D) plane of the opto-fluidic instrument 520. Additional discussion related to the SIM can be found in U.S. Provisional Application No. 63/348,879, filed Jun. 3, 2022, titled “Methods, Systems, and Devices for Sample Interface,” which is incorporated herein by reference in its entirety.

The experimental conditions that are conducive for the detection of the molecules in the sample 510 may depend on the target molecule detection technique that is employed by the opto-fluidic instrument 520. For example, in various embodiments, the opto-fluidic instrument 520 can be a system that is configured to detect molecules in the sample 510 via hybridization of probes. In such cases, the experimental conditions can include molecule hybridization conditions that result in the intensity of hybridization of the target molecule (e.g., nucleic acid) to a probe (e.g., oligonucleotide) being significantly higher when the probe sequence is complementary to the target molecule than when there is a single-base mismatch. The hybridization conditions include the preparation of the sample 510 using reagents such as washing/stripping reagents, hybridizing reagents, etc., and such reagents may be provided by the fluidics module 540.

In various embodiments, the fluidics module 540 may include one or more components that may be used for storing the reagents, as well as for transporting said reagents to and from the sample device containing the sample 510. For example, the fluidics module 540 may include reservoirs configured to store the reagents, as well as a waste container configured for collecting the reagents (e.g., and other waste) after use by the opto-fluidic instrument 520 to analyze and detect the molecules of the sample 510. Further, the fluidics module 540 may also include pumps, tubes, pipettes, etc., that are configured to facilitate the transport of the reagent to the sample device (e.g., and as such the sample 510). For instance, the fluidics module 540 may include pumps (“reagent pumps”) that are configured to pump washing/stripping reagents to the sample device for use in washing/stripping the sample 510 (e.g., as well as other washing functions such as washing an objective lens of the imaging system of the optics module 550).

In various embodiments, the ancillary module 570 can be a cooling system of the opto-fluidic instrument 520, and the cooling system may include a network of coolant-carrying tubes that are configured to transport coolants to various modules of the opto-fluidic instrument 520 for regulating the temperatures thereof. In such cases, the fluidics module 540 may include coolant reservoirs for storing the coolants and pumps (e.g., “coolant pumps”) for generating a pressure differential, thereby forcing the coolants to flow from the reservoirs to the various modules of the opto-fluidic instrument 520 via the coolant-carrying tubes. In some instances, the fluidics module 540 may include returning coolant reservoirs that may be configured to receive and store returning coolants, e.g., heated coolants flowing back into the returning coolant reservoirs after absorbing heat discharged by the various modules of the opto-fluidic instrument 520. In such cases, the fluidics module 540 may also include cooling fans that are configured to force air (e.g., cool and/or ambient air) into the returning coolant reservoirs to cool the heated coolants stored therein. In some instance, the fluidics module 540 may also include cooling fans that are configured to force air directly into a component of the opto-fluidic instrument 520 so as to cool said component. For example, the fluidics module 540 may include cooling fans that are configured to direct cool or ambient air into the system controller 530 to cool the same.

As discussed above, the opto-fluidic instrument 520 may include an optics module 550 which include the various optical components of the opto-fluidic instrument 520, such as but not limited to a camera, an illumination module (e.g., LEDs), an objective lens, and/or the like. The optics module 550 may include a fluorescence imaging system that is configured to image the fluorescence emitted by the probes (e.g., oligonucleotides) in the sample 510 after the probes are excited by light from the illumination module of the optics module 550.

In some instances, the optics module 550 may also include an optical frame onto which the camera, the illumination module, and/or the X-Y stage of the sample module 560 may be mounted.

In various embodiments, the system controller 530 may be configured to control the operations of the opto-fluidic instrument 520 (e.g., and the operations of one or more modules thereof). In some instances, the system controller 530 may take various forms, including a processor, a single computer (or computer system), or multiple computers in communication with each other. In various embodiments, the system controller 530 may be communicatively coupled with data storage, set of input devices, display system, or a combination thereof. In some cases, some or all of these components may be considered to be part of or otherwise integrated with the system controller 530, may be separate components in communication with each other, or may be integrated together. In other examples, the system controller 530 can be, or may be in communication with, a cloud computing platform.

In various embodiments, the opto-fluidic instrument 520 may analyze the sample 510 and may generate the output 590 that includes indications of the presence of the target molecules in the sample 510. For instance, with respect to the example embodiment discussed above where the opto-fluidic instrument 520 employs a hybridization technique for detecting molecules, the opto-fluidic instrument 520 may cause the sample 510 to undergo successive rounds of detectably labeled probe hybridization (e.g., using two or more sets of fluorescent probes, where each set of fluorescent probes is excited by a different color channel) and be imaged to detect target molecules in the probed sample 510. In such cases, the output 590 may include optical signatures (e.g., a codeword) specific to each gene, which allow the identification of the target molecules.

### IX. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### X. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some instances, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following example is included for illustrative purposes only and is not intended to limit the scope of the present disclosure.

### Example 1: Detection of Target Nucleic Acids Using Primary Immobilizable Probes

This example provides an exemplary method for analyzing a biological sample wherein a primary immobilizable probe that hybridizes specifically to a target nucleic acid is immobilized and detected.

In a second aspect, this example provides an exemplary method for detecting the target nucleic acid using a circularizable primary probe or probes set and performing rolling circle amplification (RCA) to generate a rolling circle amplification product (RCP) that is further anchored to a matrix, to further enhance spatial fidelity of the resulting RCP.

A biological sample (e.g., a processed or cleared biological sample, a tissue sample, a sample embedded in a hydrogel, etc.) is contacted with a primary immobilizable probe comprising a split hybridization region, a primary barcode sequence, and an attachment moiety, for example as depicted in FIG. 1A. Other configurations of primary immobilizable probes are also compatible with the methods provided herein, such as depicted in FIGS. 1B-1C. As illustrated in FIG. 4A, the attachment moiety of the primary immobilizable probe or component thereof (e.g. the first nucleic acid of the primary immobilizable probe) can comprise C6 methacrylate. However, multiple different iterations of the primary immobilizable probe with different attachment moieties disclosed herein could be used.

The first part and second part of the split hybridization region are hybridized to a first portion and second portion, respectively, of a region of interest in a target nucleic acid (e.g. as shown in FIG. 2, part i), such as an mRNA or fragment thereof. The first and second part of the split hybridization region are then ligated using the target nucleic acid (e.g. the region of interest) as template (e.g. as shown in FIG. 2, part ii) to form a ligated primary immobilizable probe. Ligation can occur with or without gap filling (e.g. using a polymerase to extend the first or second part using the region of interest as template) prior to ligation. As can be seen in FIG. 2, part ii, right panel, unhybridized first and second nucleic acid molecules of the primary immobilizable probe will generally not be ligated to one another. The sample can be washed to remove unbound probes (e.g. after hybridization and/or ligation).

Next, acrylamide monomers of a matrix-forming material are added to the biological sample, allowed to polymerize throughout the tissue and covalently incorporate the methacrylate moieties on the ligated primary immobilizable probe into the hydrogel matrix. The ligated primary immobilizable probe is now covalently attached to the matrix (e.g. as shown in FIG. 2, part ii). The ligated primary immobilizable probe may also be crosslinked to the target nucleic acid. An additional matrix polymerization step can be performed to attach generated products of probes or endogenous targets, and can be performed with different moieties. In some examples, a matrix is formed first to capture an analyte (e.g., a target nucleic acid) and then a second matrix (e.g., with a different polymer) can be formed to capture probes targeting the analyte.

Subsequent processing steps, including clearing with protease K and SDS removes the tissue components with the exception of very large (e.g., nucleus) structures, but the spatiality of the ligated primary immobilizable probe (and target nucleic acid, if crosslinked to the ligated primary immobilizable probe) is intact.

The primary barcode sequence is used to detect the ligated primary immobilizable probe at a position in the biological sample or matrix. Any of a variety of methods can be used to detect the ligated primary immobilizable probe, such as described herein or in FIGS. 3A-3D. In one example, the detection comprises rolling circle amplification. For example, a secondary circular probe is hybridized to the primary barcode region, or a secondary circularizable probe is hybridized to the primary barcode region and circularized to form a secondary circularized probe. The secondary circularizable probe is used as a template for RCA to generate an RCA product (RCP) comprising multiple copies of an RCA barcode sequence, which is then detected at spatially localized positions in the sample or matrix, for example using a detection probe and reporter oligonucleotide (e.g. as shown in FIG. 3A). An RCA primer can be provided separately to hybridize to the secondary circular or circularized probe and prime the RCA reaction. Alternatively, the overhang region of the first nucleic acid molecule to which the secondary circular or circularized probe hybridizes can serve as the RCA primer.

Optionally, the RCP can also be attached to the matrix. For example, the matrix can be a multifunctional matrix (e.g., a multifunctional hydrogel), wherein the matrix comprises acrylamide monomers for attachment to the ligated primary immobilizable probe, and a second anchoring moiety for attachment to the RCP (in one example, with methysulphone linkers included in the matrix. In some cases, a second matrix polymerization step can be performed to attach generated products. The secondary circular or circularized probe can be annealed to a thiolated primer and connected into the matrix via the methylsulphone linkers. Additionally or alternatively, the rolling circle amplification can be performed using a reaction mixture including normal dNTPs and alpha-thiol nucleotides. Thiol nucleotides can be used by Phi29 as a substrate and further connected into the matrix mesh through the orthogonal methylsulphone moieties.

In some experiments, the biological sample is contacted with an intermediate probe (e.g. the detection probe shown in FIG. 3A) that hybridizes to the RCA product. The intermediate probe further comprises one or more binding regions for fluorescently-labeled probes (e.g. the reporter oligonucleotide shown in FIG. 3A). Once a signal associated with the RCA product is detected in one probe hybridization cycle, the intermediate probe and/or fluorescently-labeled probes can be dehybridized from the RCA product (e.g., by washing). The ligated primary immobilizable probe and RCP generated therefrom (and optionally the target nucleic acid, e.g. if crosslinked to the primary immobilizable probe) remains immobilized. Multiple probe hybridization and dehybridization cycles can be performed to allow for decoding of the barcode sequence in the RCA product.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1-55. (canceled)

56. A method of analyzing a biological sample, comprising:
(a) contacting the biological sample with a primary immobilizable probe comprising:
(1) a first nucleic acid molecule comprising (i) a first part of a split hybridization region and (ii) an overhang region comprising a primary barcode sequence; and
(2) a second nucleic acid molecule comprising (i) a second part of the split hybridization region and (ii) an attachment moiety, wherein:
the first part of the split hybridization region comprises a first nucleotide sequence complementary to a first portion of a region of interest in a target nucleic acid, and
the second part of the split hybridization region comprises a second nucleotide sequence complementary to a second portion of the region of interest in the target nucleic acid;
(b) ligating the first part of the split hybridization region to the second part of the split hybridization region using the region of interest as a template, thereby forming a ligated primary immobilizable probe;
(c) using the attachment moiety, attaching the ligated primary immobilizable probe to the biological sample or to a matrix embedding the biological sample; and
(d) detecting the ligated primary immobilizable probe at a position in the biological sample and/or matrix.

57. The method of claim 56, wherein:
the primary immobilizable probe further comprises a splint oligonucleotide, wherein the splint oligonucleotide comprises a splint sequence for hybridizing to the first nucleic acid molecule and the second nucleic acid molecule; and
wherein the ligating of (b) further comprises ligating the first nucleic acid molecule and second nucleic acid molecule using the splint oligonucleotide.

58. The method of claim 56, wherein the detecting comprises using the primary barcode sequence to generate a signal corresponding to the ligated primary immobilizable probe.

59. The method of claim 56, wherein the detecting comprises:
rolling circle amplification (RCA) of a circular or circularized probe that is hybridized and/or directly or indirectly bound to the ligated primary immobilizable probe;
hybridization chain reaction (HCR) directly or indirectly on the ligated primary immobilizable probe;
linear oligonucleotide hybridization chain reaction (LO-HCR) directly or indirectly on the ligated primary immobilizable probe;
primer exchange reaction (PER) directly or indirectly on the ligated primary immobilizable probe;
assembly of branched structures directly or indirectly on the ligated primary immobilizable probe;
hybridization of a plurality of fluorescently labeled probes directly or indirectly on the ligated primary immobilizable probe;
or any combination thereof.

60. The method of claim 56, wherein the detecting comprises:
(i) hybridizing a secondary circular probe to the primary barcode sequence in the ligated primary immobilizable probe or hybridizing a secondary circularizable probe to the primary barcode sequence in the ligated primary immobilizable probe and circularizing the secondary circularizable probe to generate a secondary circularized probe,
(ii) performing rolling circle amplification (RCA) using the secondary circular or circularized probe as a template to generate a rolling circle amplification product (RCP), and
(iii) detecting the RCP at a position in the biological sample or matrix.

61. The method of claim 56, wherein the biological sample is a tissue sample and the ligated primary immobilizable probe is attached using the attachment moiety to a matrix embedding the tissue sample.

62. The method of claim 61, wherein the method comprises digesting the tissue sample before detecting the ligated primary immobilizable probe at a position in the matrix.

63. The method of claim 56, wherein the primary immobilizable probe is a circularizable probe, wherein circularizing the circularizable probe comprises ligating the first part of the split hybridization region to the second part of the split hybridization region.

64. The method of claim 56, wherein the method further comprises crosslinking the ligated primary immobilizable probe to the target nucleic acid.

65. The method of claim 60, wherein the method comprises using the overhang region of the first nucleic acid molecule as a primer for the RCA.

66. The method of claim 60, wherein the RCP comprises multiple copies of an RCP barcode sequence, wherein the method comprises hybridizing a detection probe to the RCP barcode sequence and detecting the detection probe or a product thereof, thereby detecting the RCP at the position in the biological sample or matrix.

67. The method of claim 56, wherein the target nucleic acid is DNA.

68. The method of claim 56, wherein the target nucleic acid is RNA.

69. The method of claim 56, wherein the attachment moiety is attached to an anchoring moiety in the biological sample or matrix, wherein the attachment moiety and the anchoring moiety are a ligand-ligand binding pair, or functional moieties that can react with each other.

70. The method of claim 56, wherein the method comprises performing one or more wash steps to remove unbound and/or nonspecifically bound probe molecules from the sample.

71. The method of claim 56, wherein the method comprises contacting the sample with a matrix-forming material and using the matrix-forming material to form the matrix.

72. The method of claim 69, wherein the anchoring moiety is a reactive group selected from the group consisting of acrydite, NHS ester, azide, maleimide, amine, and carboxyl groups.

73. The method of claim 56, wherein the biological sample is a formalin-fixed, paraffin-embedded (FFPE) sample or a frozen tissue sample.

74. The method of claim 56, wherein the biological sample is embedded in a matrix.

75. The method of claim 56, wherein the biological sample is cleared.
